SEQUENCE LISTING (69 sequences)

SEQ ID NO: 1 - protein, 414 aa, Yarrowia lipolytica
MDRLNNLATQLEQNPAKGLDAITSKNPDDVVITAAYRTAHTKGGKGLFKDTSSSELLASLLEGLVKESKIDPKLIGDVVCGNVLAAGAGATEHRAACLVAGIPETVPFVALNRQCSSGLMAVNDVANKIRAGQIDIGIGCGVESMSNQYGPNSVTPFSNKFQNNEEAKKCLIPMGITSENVAAKYNVSRKAQDAFAAKSYEKAAAAQAAGKFDQEILPIKTTVLDDDDNEKEVTVNKDDGIRPGVTAEKLGKLKPAFSAEGTTHAGNASQISDGAGAVLLMRRSVAEKLGQPILAKFVHCKTVGVPPELMGIGPAYAIPAVLEDLGLTVNDVDVFEINEAFASQALFSIQHCGIDESKVNPRGGAIAIGHPLGATGARQFATLLSELKESGKKVGVTSMCIGTGMGAASLVVAE

SEQ ID NO: 2 - protein, 515 aa, Tyto alba
MVSIPEYYEGKNILLTGATGFMGKVLLEKLLRSCPKVKAVYVLVRHKAGQTPEARIEEITNCKLFDRLRDEQPDFKAKIIVITSELTQPELDLSEPIKEKLIERINIIFHCAATVRFNETLRDAVQLNVTATQQLLFLAQRMKNLEVFMHVSTAYAYCNRKQIEEIVYPPPVDPKKLIDSLEWMDDGLVNDITPKLIGDRPNTYTYTKALAEYVVQQEGAKLNTAIIRPSIVGASWKEPFPGWIDNFNGPSGLFIAAGKGILRTMRASNSAVADLVPVDVVVNTTLAAAWYSGVNRPRNVMIYNCTTGGTNPFHWGEVGYHINLNFKINPLENAVRHPNCSLQSNPLLHQYWTAVSHTMPAFLLDLLLRLTGHKPWMMKTITRLHKAMMLLEYFTSNSWIWNTENMTMLMNQLNPEDKKTFNFDVRQLHWAEYMENYCMGTKKYVLNEEMSGLPAARKHLNKLRNIRYGFNTVLVILIWRIFIARSQMARNIWYFVVSLCYKFLSYFRASSTMRY

SEQ ID NO: 3 - protein, 514 aa, Marinobacter aquaeolei
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAAY

SEQ ID NO: 4 - protein, 515 aa, Mus musculus
MVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIRYGFNTILVILIWRIFIARSQMARNIWYFVVSLCYKFLSYFRASSTMRY

SEQ ID NO: 5 - protein, 15 aa, synthetic construct
GGGGSGGGGSGGGGS

SEQ ID NO: 6 - protein, 12 aa, synthetic construct
GSAGSAAGSGEF

SEQ ID NO: 7 - protein, 399 aa, Yarrowia lipolytica
MRLLIRRTGLTRPHGVQARRSTWIRLLSTEILHAELLPDRQSPHYVQESTSLSSLVWDKPLENVLIVKKPWDHNVRESLIQMASHIQRRYPRVNILVEEHVADEVQKQIGAAGVTAIHTGPGEVLRNKTDLLVTLGGDGTILHATSMFASGEVPPVLSFSLGTLGFLLPFDFKDFKTAFDMVYSSQASVVNRARLACQKMSIRKEITHLPSQSHIEHNSTHVYGNPDDYNLSPLTYAMNDINIHRGAEPHLTKLDIHVDGEFITRAIADGVTIATPTGSTAYSLSSGGSIVHPRVACILLTPICPRSLSFRPLIFPATSKICITASSESRGRGAELSVDGIAKGLVRPSDKILVESETGHNSGIWCVAKTDRDWVSGLNGLLGFNSSFGKGGEASGDVA

SEQ ID NO: 8 - protein, 366 aa, synthetic construct
MELLPDRQSPHYVQESTSLSSLVWDKPLENVLIVKKPWDHNVRESLIQMASHIQRRYPRVNILVEEHVADEVQKQIGAAGVTAIHTGPGEVLRNKTDLLVTLGGDGTILHATSMFASGEVPPVLSFSLGTLGFLLPFDFKDFKTAFDMVYSSQASVVNRARLACQKMSIRKEITHLPSQSHIEHNSTHVYGNPDDYNLSPLTYAMNDINIHRGAEPHLTKLDIHVDGEFITRAIADGVTIATPTGSTAYSLSSGGSIVHPRVACILLTPICPRSLSFRPLIFPATSKICITASSESRGRGAELSVDGIAKGLVRPSDKILVESETGHNSGIWCVAKTDRDWVSGLNGLLGFNSSFGKGGEASGDVA

SEQ ID NO: 9 - protein, 431 aa, Yarrowia lipolytica
MLTPATTRLLALSKTMSTTATRGLSTKIKVKNPIVELDGDEMTRIIWKSIKDKLILPYLDIDLKYYDLGIEYRDQTNDQVTIDAAEAIKKYQVGVKCATITPDEARVKEFGLKKMWLSPNGTIRNILGGTVFREPIVIPAVPRLVPGWKEPIIIGRHAHGDQYKAQDAVIPGAGELTLNFKPANGGDEQVIKVYTYDAPGVAMAMYNTDESITGFAYSSFNLALQKKLPLYMSTKNTILKKYDGRFKDIFQEIYDKEYKDKFDAAGIWYEHRLIDDMVAQMIKSKGGFIMALKNYDGDVQSDIVAQGFGSLGLMTSVLVTPDGKTFESEAAHGTVTRHYRQHQQGKETSTNSIASIFAWTRGLIQRGILDETPEVTKFAEALEKATVDTVDKDGIMTKDLALAGGKTDRSSYVLTEEFIDAVANRLKKDLA

SEQ ID NO: 10 - protein, 315 aa, Yarrowia lipolytica
MAAISKDYVLSPWGKAVAGAAGAVLANTLVYPLDIVKTRLQVQVKRKEGGPLPAFEEGHFEHYEGTVDALKKIYAANGLAGLYQGLPSCLLGVASTNFAYFYWYGFIRDSYIKRNPGKALSTPIELLLGAVAGALAQVFTIPVAVITTRQQTSDAKSRQGFLATAKSVVDDDGISGLWRGLKASLVLVINPSITYGSFERLRTILFKGKLHLSPGENFLLGALSKAMATIATQPMIVAKVMQQSKTKGGKQFNSFVQALVFLFKEEGILGMWKGVGPQISKGIIVQGLLFMIKDQVELFIVLLFRLMKAPTLIKG

SEQ ID NO: 11 - protein, 299 aa, Yarrowia lipolytica
MVSKLVGRTKPAGDSQKTVRIDEKVEIKEFQAHKPEIIDNTTEIPAPVPMSIAERSIADAARPPLHASPWRVFLTLFAEKGGLDKTIKLIQYTGRLLLWAAKQGWFTRHKQMMLLWKLAEMESRLNGMVSNFSQFRKIIKLGEWLGPVEDLVTTKNPLTSLAFQSELMEVINTIGDDIYCLSKIGVVKGKRLGRNGELMANWGWYGAIFINIKVGIETYQLAAKSGDEAAIWDAKLTLFKLANDFIFCTIDCLEPEGLSNIYQTVTGLASGSVGFYKLWRKISKKLDKELEEKERCKTC

SEQ ID NO: 12 - protein, 391 aa, Yarrowia lipolytica
MTDKLVKVMQKKKSAPQTWLDSYDKFLVRNAASIGSIESTLRTVSYVLPGRFNDVEIATETLYAVLNVLGLYHDTIIARAVAASPNAAAVYRPSPHNRYTDWFIKNRKGYKYASRAVTFVKFGELVAEMVAKKNGGEMARWKCIIGIEGIKAGLRIYMLGSTLYQPLCTTPYPDREVTGELLETICRDEGELDIEKGLMDPQWKMPRTGRTIPEIAPTNVEGYLLTKVLRSEDVDRPYNLLSRLDNWGVVAELLSILRPLIYACLLFRQHVNKTVPASTKSKFPFLNSPWAPWIIGLVIEALSRKMMGSWLLRQRQSGKTPTALDQMEVKGRTNLLGWWLFRGEFYQAYTRPLLYSIVARLEKIPGLGLFGALISDYLYLFDRYYFTASTL

SEQ ID NO: 13 - protein, 650 aa, Yarrowia lipolytica
MSANENISRFDAPVGKEHPAYELFHNHTRSFVYGLQPRACQGMLDFDFICKRENPSVAGVIYPFGGQFVTKMYWGTKETLLPVYQQVEKAAAKHPEVDVVVNFASSRSVYSSTMELLEYPQFRTIAIIAEGVPERRAREILHKAQKKGVTIIGPATVGGIKPGCFKVGNTGGMMDNIVASKLYRPGSVAYVSKSGGMSNELNNIISHTTDGVYEGIAIGGDRYPGTTFIDHILRYEADPKCKIIVLLGEVGGVEEYRVIEAVKNGQIKKPIVAWAIGTCASMFKTEVQFGHAGSMANSDLETAKAKNAAMKSAGFYVPDTFEDMPEVLAELYEKMVAKGELSRISEPEVPKIPIDYSWAQELGLIRKPAAFISTISDDRGQELLYAGMPISEVFKEDIGIGGVMSLLWFRRRLPDYASKFLEMVLMLTADHGPAVSGAMNTIITTRAGKDLISSLVAGLLTIGTRFGGALDGAATEFTTAYDKGLSPRQFVDTMRKQNKLIPGIGHRVKSRNNPDFRVELVKDFVKKNFPSTQLLDYALAVEEVTTSKKDNLILNVDGAIAVSFVDLMRSCGAFTVEETEDYLKNGVLNGLFVLGRSIGLIAHHLDQKRLKTGLYRHPWDDITYLVGQEAIQKKRVEISAGDVSKAKTRS

SEQ ID NO: 14 - protein, 816 aa, Yarrowia lipolytica
MFTSRVSEASTTNFIRPTARSHIHFFFAFIAATVHQLLLMLYQLLGDGYLKSFVDTGITLAQQSGLSGIVNALTSEAKLRIDKRSIIKKLLEDQENAESYFDWLKASSELDYLLGNQEWKERDECPAYDYEYVRLRLDELRHARTNNDTTRLLYLVRTTWSRNLGNLGDVKLYHNSFTGTKRLIEDYILECELALNALLAAGNDKIPDQELLTELLNTRKAFGRTALLLSGGGCLGLLHTGVLQALSDTSLLPHVISGSSAGSIMAAGLCIHKDEEHEAFITELMERDFDIFEESGNEDTVLERVSRMLKHGSLLDNRYMQDTMRELFGDMTFLEAYNRTRRILNVTVSSAGIYEMPRLLNYLTAPNVLIWSAVCASCSVPLIFNAYTLLEKEPKTGAIQTWNASSLRFIDGSVYADVPIARLSEMFNVNHFIVSQVNPHVAPFLKLTEDKANPDSVDEIYTLKLWHNFKTLVTDEVMHQLQVLYEFGIFKNLCSKMGGVLSQRYKGDITILPQVHLSELPGILTNPTAAYMKDTNRRGAQATYRKISLIRNHCAIELALDRAIHELKARMLPSKLGSGRTSPQGTFKHSQSSNQISALKPPSRHMSASSATTAHTRLRNRKSFSHARIKSDAAAVFDKEPIHETPKSSPQSSYVNLHRSASERSRRPKSAFNLGSLPTSPLYHPHLTHSMSMGGANQAPLYNPGRGSVSQNTSPGTKIPGNADPSYFDGPNNVRFHWDSDDDDVRETEFLNNMSSSSSRRVSPVQSRRASVDGLRNSVVSTATSVTDGSVSSRPSRAWESISQLFEGDENCSDSC

SEQ ID NO: 15 - protein, 691 aa, Yarrowia lipolytica
MVGYTISSKPVSVEVGPAKPGETAPRRNVIAKDAPVVFPDNDSSLTTVYKLFKKYAEINSERKAMGWRDTIDIHVETKQVTKVVDGVEKKVPKEWKYFEMGPYKWLSYKEALKLVHDYGAGLRHLGIKPKEKMHIYAQTSHRWMLSGLASLSQGIPIVTAYDTLGEEGLTRSLQETNSVIMFTDKALLSSLKVSLKKGTDLRIIIYGGDLTPDDKKAGNTEIDAIKEIVPDMKIYTMDEVVALGREHPHPVEEVDYEDLAFIMYTSGSTGVPKGVVLQHKQILASVAGVTKIIDRSIIGNTDRLLNFLPLAHIFEFVFEMVTFWWGASLGYGTVKTISDLSMKNCKGDIRELKPTIMVGVPAVWEPMRKGILGKIKELSPLMQRVFWASFAAKQRLDENGLPGGSILDSLIFKKVKDATGGCLRYVCNGGAPVSVDTQKFITTLICPMLIGCGLTETTANTTIMSPKSYAFGTIGEPTAAVTLKLIDVPEAGYFAENNQGELCIKGNVVMKEYYKNEEETKKAFSDDGYFLTGDIAEWTANGQLRIIDRRKNLVKTQNGEYIALEKLETQYRSSSYVANLCVYADQNRVKPIALVIPNEGPTKKLAQSLGVDSDDWDAVCSNKKVVKAVLKDMLDTGRSLGLSGIELLQGIVLLPGEWTPQNSYLTAAQKLNRKKIVDDNKKEIDECYEQS

SEQ ID NO: 16 - protein, 609 aa, Yarrowia lipolytica
MSDDKHTFDFIIVGGGTAGPTLARRLADAWISGKKLKVLLLESGPSSEGVDDIRCPGNWVNTIHSEYDWSYEVDEPYLSTDGEERRLCGIPRGHCLGGSSCLNTSFVIRGTRGDFDRIEEETGAKGWGWDDLFPYFRKHECYVPQGSAHEPKLIDFDTYDYKKFHGDSGPIKVQPYDYAPISKKFSESLASFGYPYNPEIFVNGGAPQGWGHVVRSTSNGVRSTGYDALVHAPKNLDIVTGHAVTKILFEKIGGKQTAVGVETYNRAAEEAGPTYKARYEVVVCCGSYASPQLLMVSGVGPKKELEEVGVKDIILDSPYVGKNLQDHLICGIFVEIKEPGYTRDHQFFDDEGLDKSTEEWKTKRTGFFSNPPQGIFSYGRIDNLLKDDPVWKEACEKQKALNPRRDPMGNDPSQPHFEIWNAELYIELEMTQAPDEGQSVMTVIGEILPPRSKGYVKLLSPDPMENPEIVHNYLQDPVDARVFAAIMKHAADVATNGAGTKDLVKARWPPESKPFEEMSIEEWETYVRDKSHTCFHPCGTVKLGGANDKEAVVDERLRVKGVDGLRVADVSVLPRVPNGHTQAFAYAVGEKAADLILADIAGKDLRPRI

SEQ ID NO: 17 - protein, 5 aa, synthetic construct
GGGGS

SEQ ID NO: 18 - protein, 8 aa, synthetic construct
GGGGGGGG

SEQ ID NO: 19 - protein, 16 aa, synthetic construct
GGGSGGGSGGGSGGGS

SEQ ID NO: 20 - protein, 46 aa, synthetic construct
AEAAAKEAAAKEAAAKEAAAKALEAEAAAKEAAAKEAAAKEAAAKA

SEQ ID NO: 21 - protein, 5 aa, synthetic construct
PAPAP

SEQ ID NO: 22 - protein, 12 aa, synthetic construct
AEAAAKEAAAKA

SEQ ID NO: 23 - protein, 5 aa, synthetic construct
EAAAK

SEQ ID NO: 24 - protein, 10 aa, synthetic construct
EAAAKEAAAK

SEQ ID NO: 25 - protein, 15 aa, synthetic construct
EAAAKEAAAKEAAAK

SEQ ID NO: 26 - protein, 20 aa, synthetic construct
EAAAKEAAAKEAAAKEAAAK

SEQ ID NO: 27 - protein, 25 aa, synthetic construct
EAAAKEAAAKEAAAKEAAAKEAAAK

SEQ ID NO: 28 - DNA, 38 bp, synthetic construct
  source 1..38: mol_type = other DNA
cgggtggtggcgcgccatggtgtctatccccgagtact

SEQ ID NO: 29 - DNA, 47 bp, synthetic construct
  source 1..47: mol_type = other DNA
cgggtggtggcgcgccatggccatccagcaggtgcaccacgctgaca

SEQ ID NO: 30 - DNA, 42 bp, synthetic construct
  source 1..42: mol_type = other DNA
cgggtggtggcgcgccatggtgtccattcccgagtactacga

SEQ ID NO: 31 - DNA, 37 bp, synthetic construct
  source 1..37: mol_type = other DNA
cgggtggtggcgcgccatggaccgacttaacaacctc

SEQ ID NO: 32 - DNA, 54 bp, synthetic construct
  source 1..54: mol_type = other DNA
ggcagcggcggaggaggaagcggcggcggaggttctatggtgtctatccccgag

SEQ ID NO: 33 - DNA, 46 bp, synthetic construct
  source 1..46: mol_type = other DNA
cttcctcctccgccgctgccacctccaccctcggcaacaaccagag

SEQ ID NO: 34 - DNA, 63 bp, synthetic construct
  source 1..63: mol_type = other DNA
acataactaattacatgaggctagcttagtatcgcatggtggaagaggctcggaagtaggaca

SEQ ID NO: 35 - DNA, 47 bp, synthetic construct
  source 1..47: mol_type = other DNA
cggcagccgatcctgccgatccctcggcaacaaccagagaagcggca

SEQ ID NO: 36 - DNA, 48 bp, synthetic construct
  source 1..48: mol_type = other DNA
ggcaggatcggctgccggttccggagaattcatggtgtctatccccga

SEQ ID NO: 37 - DNA, 94 bp, synthetic construct
  source 1..94: mol_type = other DNA
cgggtggtggcgcgccatggaccgacttaacaacctcgccacccagctcggtggaggtggcagcggcggaggaggaagcggcggcggaggttct

SEQ ID NO: 38 - DNA, 96 bp, synthetic construct
  source 1..96: mol_type = other DNA
agaacctccgccgccgcttcctcctccgccgctgccacctccaccgagctgggtggcgaggttgttaagtcggtccatggcgcgccaccacccgvv

SEQ ID NO: 39 - DNA, 89 bp, synthetic construct
  source 1..89: mol_type = other DNA
ggtggaggtggcagcggcggaggaggaagcggcggcggaggttctcgacttaacaacctcgccacccagctctaagctagccgggtggt

SEQ ID NO: 40 - DNA, 89 bp, synthetic construct
  source 1..89: mol_type = other DNA
accacccggctagcttagagctgggtggcgaggttgttaagtcgagaacctccgccgccgcttcctcctccgccgctgccacctccacc

SEQ ID NO: 41 - DNA, 54 bp, synthetic construct
  source 1..54: mol_type = other DNA
agcggcggaggaggaagcggcggcggaggttctatggtgagtttcagaggcagc

SEQ ID NO: 42 - DNA, 48 bp, synthetic construct
  source 1..48: mol_type = other DNA
cataactaattacatgaggctagcttacacccactcgtgcaggctgcc

SEQ ID NO: 43 - DNA, 48 bp, synthetic construct
  source 1..48: mol_type = other DNA
agtataagaatcattcaaaatggtgagcaagcagatcctgaagaacac

SEQ ID NO: 44 - DNA, 40 bp, synthetic construct
  source 1..40: mol_type = other DNA
cgggtggtggcgcgccatgtccaccaccgctactcgaggc

SEQ ID NO: 45 - DNA, 38 bp, synthetic construct
  source 1..38: mol_type = other DNA
cgaccaccgctagcttaagccaggtccttcttcagtct

SEQ ID NO: 46 - DNA, 42 bp, synthetic construct
  source 1..42: mol_type = other DNA
cgggtggtggcgcgccatggaactgcttcccgaccgccagtc

SEQ ID NO: 47 - DNA, 41 bp, synthetic construct
  source 1..41: mol_type = other DNA
cgaccaccgctagcttagagcttagcagcaacatcgcctga

SEQ ID NO: 48 - DNA, 41 bp, synthetic construct
  source 1..41: mol_type = other DNA
cgaccaccgctagcttacagcttggaagcaacatcgcctga

SEQ ID NO: 49 - DNA, 46 bp, synthetic construct
  source 1..46: mol_type = other DNA
gaggcgtcaggcgatgttgcttctaaggccctgggcaagggagtga

SEQ ID NO: 50 - DNA, 42 bp, synthetic construct
  source 1..42: mol_type = other DNA
gagtataagaatcattcaaaatggtgagtttcagaggcagca

SEQ ID NO: 51 - DNA, 45 bp, synthetic construct
  source 1..45: mol_type = other DNA
cataactaattacatgaggctagcttatcccttgatcaaggtggg

SEQ ID NO: 52 - DNA, 44 bp, synthetic construct
  source 1..44: mol_type = other DNA
cgggtggtggcgcgccatggtgtctaagctggttggacggacga

SEQ ID NO: 53 - DNA, 41 bp, synthetic construct
  source 1..41: mol_type = other DNA
cgaccaccgctagcttactagcaggtcttgcatcgctcctt

SEQ ID NO: 54 - DNA, 47 bp, synthetic construct
  source 1..47: mol_type = other DNA
cgggtggtggcgcgccatgacggacaagctggtcaaggtgatgcaga

SEQ ID NO: 55 - DNA, 46 bp, synthetic construct
  source 1..46: mol_type = other DNA
acataactaattacatgaggctagcttagagagtagaggcggtgaa

SEQ ID NO: 56 - DNA, 36 bp, synthetic construct
  source 1..36: mol_type = other DNA
cgggtggtggcgcgccatgatttctgctattcgtcc

SEQ ID NO: 57 - DNA, 34 bp, synthetic construct
  source 1..34: mol_type = other DNA
cgaccaccgctagcttagagcttgaggccaacga

SEQ ID NO: 58 - DNA, 44 bp, synthetic construct
  source 1..44: mol_type = other DNA
cgggtggtggcgcgccatgttcacctccagagtttccgaagcaa

SEQ ID NO: 59 - DNA, 42 bp, synthetic construct
  source 1..42: mol_type = other DNA
cgaccaccgctagcttagcacgagtcagaacagttctcgtcc

SEQ ID NO: 60 - DNA, 45 bp, synthetic construct
  source 1..45: mol_type = other DNA
ttccgggtcggcgcaggttgacgtacagcacatacgaggatcggt

SEQ ID NO: 61 - DNA, 45 bp, synthetic construct
  source 1..45: mol_type = other DNA
aacttgctatttctagctctaaaacaccgatcctcgtatgtgctg

SEQ ID NO: 62 - DNA, 62 bp, synthetic construct
  source 1..62: mol_type = other DNA
ggcagcggcggaggaggaagcggcggcggaggttctatggccatccagcaggtgcaccacgc

SEQ ID NO: 63 - DNA, 42 bp, synthetic construct
  source 1..42: mol_type = other DNA
cgaccaccgctagcttagtaagcggccttctttcgctgtcgg

SEQ ID NO: 64 - DNA, 62 bp, synthetic construct
  source 1..62: mol_type = other DNA
ggcagcggcggaggaggaagcggcggcggaggttctatggtgtccattcccgagtactacga

SEQ ID NO: 65 - DNA, 42 bp, synthetic construct
  source 1..42: mol_type = other DNA
cgaccaccgctagcttagtatcgcatggtggaagaggctcgg

SEQ ID NO: 66 - DNA, 1548 bp, synthetic construct
  source 1..1548: mol_type = other DNA
atggtgtctatccccgagtactacgagggaaagaacattctgctgaccggagctaccggtttcatgggcaaggtgctgctggagaagctgctgcgatcctgtcccaaggtcaaggccgtctacgtgctggtgcgacacaaggctggacagacccctgaggctcgaattgaggagattaccaactgcaagctgttcgaccgactgcgagacgagcagcccgacttcaaggctaagatcattgtgatcacctctgagctgacccagcctgagctggacctgtctgagcccattaaggagaagctgatcgagcgaattaacatcattttccactgtgccgctaccgtccgattcaacgagaccctgcgagacgctgtgcagctgaacgtcaccgctacccagcagctgctgttcctggctcagcgaatgaagaacctggaggtgttcatgcacgtctctaccgcttacgcctactgcaaccgaaagcagatcgaggagattgtgtaccctcctcccgtcgaccccaagaagctgatcgactccctggagtggatggacgacggactggtgaacgacatcacccccaagctgattggtgaccgacccaacacctacacctacaccaaggctctggccgagtacgtggtccagcaggaaggcgctaagctgaacaccgccatcattcgaccctctatcgtgggtgcttcctggaaggagcccttccccggctggattgacaacttcaacggaccctctggtctgttcatcgccgctggaaagggcattctgcgaaccatgcgagcctctaactctgctgtggctgacctggtgcctgtggacgtggtcgtgaacaccaccctggctgctgcttggtactctggtgtgaaccgaccccgaaacgtcatgatttacaactgtaccaccggcggaaccaaccctttccactggggcgaggtcggataccacatcaacctgaacttcaagattaaccccctggagaacgccgtccgacaccccaactgctctctgcagtccaaccccctgctgcaccagtactggaccgctgtgtctcacaccatgcctgccttcctgctggacctgctgctgcgactgaccggacacaagccctggatgatgaagaccatcacccgactgcacaaggctatgatgctgctggagtacttcacctctaactcctggatttggaacaccgagaacatgaccatgctgatgaaccagctgaaccccgaggacaagaagaccttcaacttcgacgtgcgacagctgcactgggccgagtacatggagaactactgtatgggcaccaagaagtacgtcctgaacgaggagatgtctggactgcccgctgcccgaaagcacctgaacaagctgcgaaacatccgatacggcttcaacaccgtgctggtcatcctgatttggcgaatcttcattgctcgatcccagatggcccgaaacatttggtacttcgtcgtgtctctgtgctacaagttcctgtcctacttccgagcctcttccaccatgcgatactaa

SEQ ID NO: 67 - DNA, 1545 bp, synthetic construct
  source 1..1545: mol_type = other DNA
atggccatccagcaggtgcaccacgctgacacctcttcctctaaggtcctgggtcagctgcgaggcaagcgagtgctgattaccggcaccaccggattcctgggaaaggtggtcctggagcgactgatccgagccgtccccgacatcggcgctatttacctgctgattcgaggaaacaagcgacaccccgacgcccgatcccgattcctggaggagatcgctacctcctctgtgttcgaccgactgcgagaggccgactctgagggtttcgacgctttcctggaggagcgaatccactgcgtgaccggagaggtgaccgaggctggtttcggaattggtcaggaagactaccgaaagctggccaccgagctggacgctgtcattaactccgccgcttctgtgaacttccgagaggagctggacaaggccctggctatcaacaccctgtgtctgcgaaacattgccggaatggtcgacctgaaccccaagctggctgtgctgcaggtctccacctgctacgtgaacggaatgaactccggtcaggtcaccgagtctgtgattaagcctgctggagaggctgtccctcgatctcccgacggtttctacgagatcgaggagctggtccgactgctgcaggacaagattgaggacgtgcaggcccgatactccggcaaggtcctggagcgaaagctggtggacctgggtattcgagaggctaaccgatacggctggtctgacacctacaccttcaccaagtggctgggcgagcagctgctgatgaaggccctgaacggacgaaccctgaccatcctgcgaccctccatcattgagtccgctctggaggagcctgctcctggttggattgagggagtgaaggtcgccgacgctatcattctggcctacgctcgagagaaggtgaccctgttccccggaaagcgatccggcatcattgacgtgatccccgtcgacctggtggccaactccatcattctgtctctggctgaggctctgggagagcctggacgacgacgaatttaccagtgttgctccggcggaggtaaccccatctctctgggagagttcattgaccacctgatggccgagtccaaggctaactacgccgcttacgaccacctgttctaccgacagccctctaagcccttcctggccgtcaaccgagctctgttcgacctggtcatctccggagtgcgactgcccctgtctctgaccgaccgagtgctgaagctgctgggtaactcccgagacctgaagatgctgcgaaacctggacaccacccagtctctggccaccatcttcggattctacaccgctcccgactacattttccgaaacgacgagctgatggccctggctaaccgaatgggagaggtcgacaagggtctgttccccgtggacgcccgactgatcgactgggagctgtacctgcgaaagattcacctggccggcctgaaccgatacgctctgaaggagcgaaaggtgtactctctgaagaccgcccgacagcgaaagaaggccgcttactaa

SEQ ID NO: 68 - DNA, 1548 bp, synthetic construct
  source 1..1548: mol_type = other DNA
atggtgtccattcccgagtactacgagggcaagaacatcctgctgaccggtgctaccggcttcctgggaaaggtgctgctggagaagctgctgcgatcttgtccccgagtcaactccgtctacgtgctggtccgacagaaggccggacagacccctcaggagcgagtcgaggagattctgtcttccaagctgttcgaccgactgcgagacgagaaccccgacttccgagagaagatcattgctatcaactctgagctgacccagcccaagctggccctgtccgaggaagacaaggagatcattatcgactctaccaacgtgattttccactgtgccgctaccgtccgattcaacgagaacctgcgagacgctgtgcagctgaacgtcattgccacccgacagctgatcctgctggcccagcagatgaagaacctggaggtgttcatgcacgtctccaccgcttacgcctactgcaaccgaaagcacattgacgaggtggtctaccctcctcccgtggaccccaagaagctgatcgactctctggagtggatggacgacggtctggtcaacgacattacccccaagctgatcggcgaccgacccaacacctacatttacaccaaggctctggccgagtacgtggtccagcaggaaggcgctaagctgaacgtggccattgtccgaccctctatcgtgggagcttcctggaaggagcctttccctggatggatcgacaacttcaacggtccctccggcctgttcattgccgctggcaagggaatcctgcgaaccatgcgagcctctaacaacgctctggctgacctggtgcctgtggacgtggtcgtgaacacctccctggctgctgcttggtactctggtgtgaaccgaccccgaaacattatggtctacaactgcaccaccggatccaccaaccccttccactggggtgaggtggagtaccacgtcatctctaccttcaagcgaaaccccctggagcaggctttccgacgacccaacgtgaacctgacctccaaccacctgctgtaccactactggattgccgtctctcacaaggctcccgccttcctgtacgacatctacctgcgaatgaccggacgatctccccgaatgatgaagaccattacccgactgcacaaggctatggtgttcctggagtacttcacctctaactcctgggtgtggaacaccgacaacgtcaacatgctgatgaaccagctgaaccccgaggacaagaagaccttcaacattgacgtgcgacagctgcactgggccgagtacatcgagaactactgtatgggaaccaagaagtacgtcctgaacgaggagatgtctggtctgcctgctgctcgaaagcacctgaacaagctgcgaaacattcgatacggcttcaacaccatcctggtgattctgatctggcgaattttcatcgctcgatctcagatggcccgaaacatctggtacttcgtcgtgtctctgtgctacaagttcctgtcctacttccgagcctcttccaccatgcgatactaa

SEQ ID NO: 69 - DNA, 1101 bp, synthetic construct
  source 1..1101: mol_type = other DNA
atggaactgcttcccgaccgccagtcgccccactacgtccaggagtcgacctctctgtcatctctggtgtgggacaagcctctggaaaacgttctgatcgtcaaaaaaccctgggaccacaatgtgcgcgagtcgctcatccagatggcatctcacatccagcgccggtacccccgagtcaacattctggtggaggaacatgtggccgacgaggtccagaagcagattggagccgcaggcgtgaccgccatccacacggggccaggagaggtgctgagaaacaagacggatctgctcgtgactctgggaggcgacggaactattctacatgccacctccatgtttgcttccggagaagtgccgccggtgctgtccttttcgctggggactctgggtttcctgctgccgtttgatttcaaggacttcaaaactgcattcgacatggtgtactcgtcgcaggcctcggtggtcaaccgcgcccgcctagcatgtcagaaaatgtccattcgcaaggaaatcacccacttgccctcccaatcgcacattgaacacaactcaacccatgtctacggcaatcccgacgactacaatcttagcccactaacctacgccatgaacgacatcaacatccaccgtggagctgagccgcatctcaccaagctcgacatccacgttgacggcgagttcatcacccgagccattgctgacggtgtcaccatcgccacacccacgggctccacggcctactcgctgtcgtctggcggctccattgtgcatccccgagtcgcctgcattctgctgacccccatctgtccgcgatcgctgtcattccggcctctcattttcccagccacctccaaaatatgcatcaccgcctcgtccgaatctcgaggtagaggcgccgagctgtctgtcgacggaatcgccaagggtctggttcgacccagcgacaagattctggtcgaaagcgaaaccggccacaactcgggcatctggtgcgtggccaagacagacagagactgggtcagtggcctcaacgggttactgggcttcaatagcagttttggcaagggcggggaggcgtcaggcgatgttgcttaa

(19) United States
(12) Patent Application Publication
(10) Pub. No.: US 2026/0043055 A1
(43) Pub. Date: Feb. 12, 2026

(54) PRODUCTION OF FATTY ALCOHOLS IN PEROXISOME OF YARROWIA LIPOLYTICA

(71) Applicants: SIVACHANDIRAN, Fnu, Newark, DE (US); BLENNER, Mark, Newark, DE (US); SPAGNUOLO, Michael, Newark, DE (US)

(72) Inventors: Fnu Sivachandiran, Newark, DE (US); Mark Blenner, Newark, DE (US); Michael Spagnuolo, Newark, DE (US)

(73) Assignee: University of Delaware, Newark, DE (US)

(21) Appl. No.: 18/998,993

(22) PCT Filed: Jul. 28, 2023
(86) PCT No.: PCT/US2023/071236

§ 371 Date: Jan. 27, 2025

Related U.S. Application Data
(60) Provisional application No. 63/392,900, filed on Jul. 28, 2022.

(51) Int. Cl. C12P 7/64; C12N 9/00; C12N 9/02; C12N 9/04; C12N 9/10; C12N 9/12; C12N 15/52; C12N 15/81

(52) U.S. Cl. CPC C12P 7/64; C12N 9/0006; C12N 9/0008; C12N 9/1096; C12N 9/1205; C12N 9/93; C12N 15/52; C12N 15/815; C12Y 101/0302; C12Y 102/01084; C12Y 206/01007; C12Y 207/01086; C12Y 602/01003; C12N 2800/102

(57) ABSTRACT

The invention provides a recombinant Yarrowia lipolytica. The recombinant Yarrowia lipolytica comprises a heterologous polynucleotide encoding a fusion protein. The fusion protein comprises a first amino acid sequence and a second amino acid sequence. The first amino acid may be homologous to the amino acid sequence of 3-ketoacyl CoA thiolase (3KAT), The second amino acid sequence may be homologous to the amino acid sequence of a fatty acyl-CoA reductase (FAR). Also provided is a method for producing one or more fatty alcohols by the recombinant Yarrowia lipolytica and a method for preparing the recombinant Yarrowia lipolytica.

### CROSS-REFERENCE TO RELATED APPLICATION

This application claims priority to U.S. Provisional Application No. 63/392,900, filed Jul. 28, 2022, and the contents of which are incorporated herein by reference in their entireties for all purposes.

### REFERENCE TO U.S. GOVERNMENT SUPPORT

This invention was made with government support under Grant No, CBET1706134 from the National Science Foundation (NSF) and Grant No. 80NSSC22K0118 from the National Aeronautics and Space Administration (NASA). The United States has certain rights in the invention.

### REFERENCE TO SEQUENCE LISTING

The present application is being filed along with a Sequence Listing in electronic format. The Sequence Listing is provided as a file entitled UOD-564WO_SequenceUsting.XML created on Jul. 28, 2023, which is 73.1 KB in size.

### FIELD OF THE INVENTION

This invention relates generally to production of fatty alcohols by microorganisms, for example, oleaginous yeast such as Yarrowia lipolytica.

### BACKGROUND OF THE INVENTION

Fatty alcohols are an oleochemical with a wide range of applications in cosmetics, detergents and pharmaceutical industries, Currently, fatty alcohols are produced by oligomerization of fossil fuel derived ethylene followed by hydrogenation or from hydrogenation of non-sustainable palm oils, Therefore, the development of a sustainable process to make fatty alcohols is needed. Fatty alcohols can be produced biologically in one of two ways, either by reduction of a fatty acyl-CoA or by the combined action of a carboxylic acid reductase and alcohol dehydrogenase on fatty acids. Based on the toxic effects of free fatty acids, and the availability of acyl-CoA, many studies favor the use of fatty acyl-CoA reductases (FARs) to catalyze fatty alcohol production.

Yarrowia lipolytica (Y. lipolytica) is an oleaginous yeast that has naturally evolved to produce high lipid content directed through the strong flux through the fatty acid synthesis pathway. Therefore, Yarrowia species are considered a promising host for production of fatty alcohols. Several previous studies have investigated Y. lipolytica for fatty alcohol production. The TaFAR from Tyto alba was expressed in Y. lipolytica, resulting in 690 ng/L of hexadecanol from flask culture. Similarly, overexpression of MaACR from Marinobacter aquaeolei VT8 combined with a standard push-pull-block strategy achieved 557 mg/L of fatty alcohol in flask culture. More recently, combining high lipid flux strains of Y. lipolytica with overexpression of MhFAR from Marinobacter hydrocarbonoclasticus, achieved 5.8 g/L of fatty alcohols from a bioreactor, in which Oleyl alcohol (C18:1) was the major constitute. In these studies, fatty alcohol production occurs in the cytosol; however, there are numerous competing pathways for fatty acyl-CoA and that potential toxicity of fatty alcohols may limit the titer of the produced fatty alcohols.

Compartmentalizing product pathways and colocalizing sequential reactions (sometimes controversially called substrate channeling) have been successful metabolic engineering strategies. The peroxisome is an organelle where beta oxidation occurs in yeast. During beta oxidation, fatty acyl-CoAs are shortened two carbons per cycle. Several studies have examined the potential to use peroxisomes for compartmentalizing metabolic pathways. In Saccharomyces cerevisiae, localizing exogenous MaACR to peroxisomes resulted in 193 mg/L of fatty alcohols. Localizing TaFAR to the peroxisomes using PTS1 along with additional engineering strategies led to production of over 1.3 g/L of fatty alcohols in fed-batch fermentation. Further, in methylotrophic yeast Ogataea polymorpha, the coupling of fatty alcohol biosynthesis and methanol utilization in peroxisomes has been shown to significantly boost the fatty alcohol production by 3.9 fold compared to cytosolic fatty alcohol production.

There remains a need for improving fatty alcohol production by targeting the production to peroxisomes of microorganisms, especially Yarrowia lipolytica.

### SUMMARY OF THE INVENTION

The present invention relates to a recombinant Yarrowia lipolytica for producing fatty alcohols. The inventors have surprisingly discovered a recombinant Yarrowia lipolytica expressing a fusion protein of 3-ketoacyl CoA thiolase (3KAT) and a fatty acyl-CoA reductase (FAR) in peroxisomes and generating fatty alcohols in the peroxisomes.

A recombinant Yarrowia lipolytica is provided. The recombinant Yarrowia lipolytica comprises a heterologous polynucleotide encoding a fusion protein. The fusion protein comprises a first amino acid sequence at least 80% identical to the amino acid sequence of 3-ketoacyl CoA thiolase (3KAT) (SEQ ID NO: 1) and a second amino acid sequence at least 80% identical to the amino acid sequence of a fatty acyl-CoA reductase (FAR). The first amino acid sequence may be the amino acid sequence of the 3KAT (SEQ ID NO: 1) and the second amino acid sequence may be the amino acid sequence of the FAR. The FAR may be selected from the group consisting of TaFAR (SEQ ID NO: 2), MaACR (SEQ ID NO: 3) and MmFAR (SEQ ID NO: 4). The fusion protein may further comprise a linker between the first amino acid sequence and the second amino acid sequence.

The recombinant Yarrowia lipolytica may have knockout of fatty acyl-CoA synthetase (FAA1) (SEQ ID NO: 15), fatty alcohol oxidase (FAO1) (SEQ ID NO: 16), or a combination thereof.

The recombinant Yarrowia lipolytica may express the fusion protein in one or more peroxisomes in the recombinant Yarrowia lipolytica and generate one or more fatty alcohols in the one or more peroxisomes. The one or more fatty alcohols may comprise a C16:0 fatty alcohol, a C18:0 fatty alcohol, or a combination thereof. The recombinant Yarrowia lipolytica may further express a recombinant NADH kinase having an amino acid sequence at least 80% identical to SEQ ID NO: 8 in the one or more peroxisomes. The recombinant Yarrowia lipolytica may overexpress an endogenous protein selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), and triacylglycerol lipase (TGL4) (SEQ ID NO: 14).

A method for producing one or more fatty alcohols is also provided. The production method comprises growing a recombinant Yarrowia lipolytica in a culture medium. The recombinant Yarrowia lipolytica comprises a heterologous polynucleotide encoding a fusion protein. The fusion protein comprises a first amino acid sequence at least 80% identical to the amino acid sequence of 3-ketoacyl CoA thiolase (3KAT) (SEQ ID NO: 1) and a second amino acid sequence at least 80% identical to the amino acid sequence of a fatty acyl-CoA reductase (FAR). The production method further comprises expressing the fusion protein in one or more peroxisomes in the recombinant Yarrowia lipolytica, and generating one or more fatty alcohols by the recombinant Yarrowia lipolytica. The one or more fatty alcohols are produced in the culture medium. The first amino acid sequence may be the amino acid sequence of the 3KAT (SEQ ID NO: 1) and the second amino acid sequence may be the amino acid sequence of the FAR. The FAR may be selected from the group consisting of TaFAR (SEQ ID NO: 2), MaACR (SEQ ID NO: 3) and MmFAR (SEQ ID NO: 4). The fusion protein may further comprise a linker between the first amino acid sequence and the second amino acid sequence. The recombinant Yarrowia lipolytica may have knockout of fatty acyl-CoA synthetase (FAA1) (SEQ ID NO: 15), fatty alcohol oxidase (FAO1) (SEQ ID NO: 16), or a combination thereof.

The production method may further comprise expressing a recombinant NADIH kinase in the one or more peroxisomes by the recombinant Yarrowia lipolytica, and the recombinant NADH kinase may comprise an amino acid sequence at least 80% identical to SEQ ID NO: 8.

The production method may further comprise overexpressing an endogenous protein selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), and triacylglycerol lipase (TGL4) (SEQ ID NO: 14).

The production method may further comprise adding dodecane to the culture medium before the growing, and the culture medium may become biphasic.

According to the production method, the recombinant Yarrowia lipolytica may be grown in a continuous culture or in fed-batch fermentation. The culture medium may comprise the one or more fatty alcohols at a concentration of 0.1-10 g per liter of the culture medium.

According to the production method, the one or more fatty alcohols may comprise a C16:0 fatty alcohol, a C18:0 fatty alcohol or a combination thereof. 85-95% of the one or more fatty alcohols may be the C16:0 fatty alcohol.

A method for preparing a recombinant Yarrowia lipolytica is further provided. The preparation method comprises introducing a heterologous polynucleotide into Yarrowia lipolytica. The heterologous polynucleotide encodes a fusion protein. The fusion protein comprises a first amino acid sequence at least 80% identical to the amino acid sequence of 3-ketoacyl CoA thiolase (3KAT) (SEQ ID NO:1) and a second amino acid sequence at least 80% identical to the amino acid sequence of a fatty acyl-CoA reductase (FAR), The first amino acid sequence may be the amino acid sequence of the 3KAT (SEQ ID NO: 1) and the second amino acid sequence may be the amino acid sequence of the EAR. The FAR may be selected from the group consisting of TaFAR (SEQ ID NO: 2), MaACR (SEQ ID NO: 3) and MmFAR (SEQ ID NO: 4). The fusion protein may further comprise a linker between the first amino acid sequence and the second amino acid sequence. The recombinant Yarrowia lipolytica may express the fusion protein in one or more peroxisomes in the recombinant Yarrowia lipolytica and generate one or more fatty alcohols in the one or more peroxisomes. The one or more fatty alcohols may comprise a C16:0 fatty alcohol, a C18:0 fatty alcohol, or a combination thereof.

The preparation method may further comprise knocking out fatty acyl-CoA synthetase (FAA1) (SEQ ID NO: 15), fatty alcohol oxidase (FAO1) (SEQ ID NO: 16), or a combination thereof in the recombinant Yarrowia lipolytica.

According to the preparation method, the recombinant Yarrowia lipolytica may express a recombinant NADH kinase having an amino acid sequence at least 80% identical to SEQ ID NO: 8 in the one or more peroxisomes in the recombinant Yarrowia lipolytica. The recombinant Yarrowia lipolytica may overexpress an endogenous protein selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), and triacylglycerol lipase (TGL4) (SEQ ID NO: 14).

For each preparation method according to the present invention, a recombinant Yarrowia lipolytica prepared by the method is provided.

### BRIEF DESCRIPTION OF THE DRAWINGS

FIGS. 1a-d show localization and production of fatty alcohols in peroxisomes by 3KAT fusion: (a) Schematic diagram of localization of fatty alcohol biosynthetic enzyme FAR in a peroxisome using 3KAT enzyme fusion strategies; (b) Construction of fusion plasmids using 3KAT and PTS2 signal tag; (c) Visualization of enzyme localization using fluorescence microscopy; and (d) Fatty alcohol titer of engineered strains in shake flasks after 7 days of flask fermentation.

FIGS. 2a-b show auxiliary enzyme overexpression in peroxisome: (a) Schematic diagram of IDP3 overexpression to supply NADPH to TAFAR in peroxisome; and (b) Fatty alcohol production from of DP3 overexpressing strain and control strain,

FIGS. 3a-c show redox engineering strategies in peroxisome: (a) Schematic illustration of NADH kinase (pos5) localization; (b) Fatty alcohol titer of engineered strains in shake flasks after 7 days of culture; and (c) Secreted Fatty acid titer quantified from dodecane overlay at different time points of culture,

FIGS. 4a-c show: additional engineering to improve fatty alcohol production: (a) Schematic diagram of peroxisome biogenesis by PEX11 and PEX16; (b) Overexpression of ACL and TGL4 to increase the free fatty acid accumulation; and (c) Fatty alcohol production from various engineering strains,

FIGS. 5a-b show fermentation of strain FS114 for fatty alcohol production in bioflo 320: (a) Total fatty alcohol production and specific productivity, Arrow represent the glucose spike 80 g at 72 h and 144 h; and (b) Measurement of glucose concentration and citrate accumulation throughout the fermentation.

### DETAILED DESCRIPTION OF THE INVENTION

The present invention relates to a recombinant Yarrowia lipolytica expressing a fusion protein comprising 3-ketoacyl CoA thiolase (3KAT) and a fatty acyl-CoA reductase (FAR), also referred to as the 3KAT/FAR fusion protein, in its peroxisomes and production of fatty alcohols in the peroxisomes. The present invention is based on inventors' surprising discovery of expression of the 3KAT/FAR fusion protein in peroxisomes of a recombinant Yarrowia lipolytica enables fatty alcohol production in the peroxisomes and improves fatty alcohol production by the recombinant Yarrowia lipolytica.

The inventors have targeted fatty alcohol production to peroxisomes, where acyl-CoA flux is directed during beta-oxidation, and with fewer competing pathways. After media optimization, FARs from bacterial and mammalian sources were screened using canonical peroxisome targeting sequences, as well as an enzyme fusion strategy to physically colocalize FAR next to 3-ketoacyl-CoA thiolase (3KAT) in a peroxisome. 3KAT fusion resulted in nearly double the titer of fatty alcohols irrespective of overexpression of any FAR. The inventors then systematically harnessed the subcellular organelle and increased the peroxisome organelle capacity for fatty alcohol production by increasing peroxisome numbers and increasing NADPH availability. The highest titer achieved in shake flask culture was over 1.6 g/L of fatty alcohol Finally, the inventors scaled-up and demonstrated compartmentalized production of fatty alcohols at 2.77 g/L in a 5 L bioreactor, including C16:0 hexadecanol at 2.53 g/L.

In one study, the inventors engineered the peroxisomes of Yarrowia lipolytica for compartmentalized production of fatty alcohols. The inventors localized and spatially organized the fatty alcohol biosynthetic enzymes such as FARs that are not naturally localized in peroxisomes into peroxisomes using an enzyme fusion strategy. The inventors systematically identified the redox bottlenecks in the peroxisomes and increased the localized redox power supply by overexpressing NADH kinase and auxiliary enzyme IDP3 in peroxisomes. Next, the inventors modulated the biogenesis of peroxisomes and increased the precursor fatty acid flux towards production of fatty alcohols. Finally, the inventors successfully scaled up fatty alcohol producing Y. lipolytica strain from a 12 mL flask culture to a 5 L bioreactor using bi-phasic extractive fermentation for 10 days and achieved the highest C16:0 hexadecanol of 2.53 g/L to date.

The terms “recombinant Yarrowia lipolytica” and “engineered Yarrowia lipolytica” are used herein interchangeably and refer to a naturally occurring Yarrowia lipolytica that has been genetically modified. The naturally occurring Yarrowia lipolytica before modification is referred to as a control Yarrowia lipolytica of the recombinant Yarrowia lipolytica.

The term “endogenous” as used herein refers to the source of a protein or gene that is naturally occurring in a cell.

The term “exogenous” as used herein refers to the source of a protein or gene that is not naturally occurring in a cell.

The term “linker” as used herein refers to a short sequence of amino acids. The link may have a length of about 5˜25 amino acids.

The term “flexible linker” used herein refers to a linker that is a Glycine Serine (GS) rich linker, containing any combination of predominately glycine and serine residues.

The term “rigid linker” as used herein refers to a linker that is a linker having several hydrophobic or bulky residues, for example, G, S, A, G, S, A, A, G, S, G, E, and F.

The term “knockout” as used herein refers to disruption of an endogenous gene in a cell and thus reduction or elimination of expression of its corresponding endogenous protein in the cell.

The term “overexpressing” as used herein refers to increased transcriptional expression level of an endogenous gene or increased expression of an endogenous protein in a recombinant cell above that in a control cell. The recombinant cell may comprise an increased copy number of the endogenous gene and/or addition of a stronger promoter as compared to that in the control cell.

The term “bi-phasic extractive fermentation” as used herein refers to a fermentation process in which a solvent, for example, dodecane is added to the top of a culture of cells to extract a metabolite, for example, a fatty alcohol, produced and secreted by the cells. The cell may comprise one or more peroxisomes. The cell may be a yeast, for example, Yarrow lipolytica. The cell may be naturally occurring. The cell may be recombinant or engineered.

The term “fed-batch fermentation” as used herein refers to an operational technique in biomanufacturing where one or more nutrients are fed to a bioreactor during cultivation and a product remains in the bioreactor until the end of the cultivation.

The present invention provides a recombinant Yarrowia lipolytica. The recombinant Yarrowia lipolytica comprises a heterologous polynucleotide encoding a fusion protein. The fusion protein comprises a first amino acid sequence and a second amino acid sequence.

The first amino acid sequence may be at least about 60%, 70%, 80%, 90%, 95%, 99% or 100% identical to the amino acid sequence of 3-ketoacyl CoA thiolase (3KAT) (SEQ ID NO: 1), also known as POT1. The first amino acid sequence may be the amino acid sequence of 3KAT.

The second amino acid sequence may be at least about 60%, 70%, 80%, 90%, 95%, 99% or 100% identical to the amino acid sequence of a fatty acyl-CoA reductase (FAR). The FAR is an exogenous protein to Yarrowia lipolytica. The FAR may be TaFAR (SEQ ID NO: 2), MaACR (SEQ ID NO: 3) or MmFAR (SEQ ID NO: 4). The second amino acid sequence may be at least about 60%, 70%, 80%, 90%, 95%, 99% or 100% identical to the amino acid sequence of TaFAR, MaACR or MmFAR. For example, the second amino acid sequence may be the amino acid sequence of TaFAR, MaACR or MmFAR.

In the fusion protein, the first amino acid sequence may be at the N-terminus or C-terminus of the second amino acid sequence. The fusion protein may comprise 3KAT and a FAR, wherein the 3KAT may be at the N-terminus or C-terminus of the FAR. The FAR may be TaFAR, MaACR or MmFAR. The fusion protein may consist of 3KAT and a FAR, wherein the 3KAT may be at the N-terminus or C-terminus of the FAR. The FAR may be TaFAR, MaACR or MmFAR.

The fusion protein may further comprise a linker between the first amino acid sequence and the second amino acid sequence. The first amino acid sequence may be at the N-terminus of the linker, and the linker may be at the N-terminus of the second amino acid sequence. The first amino acid sequence may be at the C-terminus of the linker, and the linker may be at the C-terminus of the second amino acid sequence. The linker may comprise about 5-25 amino acids. The linker may be flexible, for example, consisting of the amino acid sequence of GGGGSGGGGSGGCGS (SEQ ID NO: 5), GGGGS (SEQ ID NO: 17), GGGGGGGG (SEQ ID NO: 18), or GGGSGGGSGGGSGGGS (SEQ ID NO: 19), The linker may be rigid, for example, consisting of the amino acid sequence of GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22) or (EAAAK), wherein n may be between 1 and 5, for example, EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) and EAAAKEAAAKEAAAKEAAAKEAAAK (SEQ ID NO: 27).

The fusion protein may comprise 3KAT, a FAR and a linker, wherein the 3KAT may be at the N-terminus of the linker and the linker may be at the N-terminus of the FAR. The fusion protein may consist of 3KAT, a FAR and a linker, wherein the 3KAT may be at the N-terminus of the linker and the linker may be at the N-terminus of the FAR. The FAR may be TaFAR, MaACR or MmFAR. The linker may consist of GGGGSGGGGSGGGGS (SEQ ID NO: 5), GGGGS (SEQ ID NO: 17), GGGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

The fusion protein may comprise 3KAT, a FAR and a linker, wherein the 3KAT may be at the C-terminus of the linker and the linker may be at the C-terminus of the FAR. The fusion protein may consist of 3KAT, a FAR and a linker, wherein the 3KAT may be at the C-terminus of the linker and the linker may be at the C-terminus of the FAR. The FAR may be TaFAR, MaACR or MmFAR. The linker may consist of GGGGSGGGGSGGGGS (SEQ ID NO: 5), GGGGS (SEQ ID NO: 17), GGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

The recombinant Yarrowia lipolytica may have knockout of one or more endogenous proteins, for example, fatty acyl-CoA synthetase (FAA1) (SEQ ID NO: 15), fatty alcohol oxidase (FAO1) (SEQ ID NO: 16), or a combination thereof.

The recombinant Yarrowia lipolytica may express the fusion protein in one or more peroxisomes in the recombinant Yarrowia lipolytica and produce one or more fatty alcohols. The one or more fatty alcohols may be generated in the one or more peroxisomes. The one or more fatty alcohols may be secreted by the recombinant Yarrowia lipolytica. The one or more fatty alcohols may comprise a C8:0 fatty alcohol, C10:0 fatty alcohol, C12:0 fatty alcohol, C16:0 fatty alcohol, C18:0 fatty alcohol, C18:1 fatty alcohol, or combination thereof. The C16:0 fatty alcohol may be hexadecanol, cetanol, cetyl alcohol, ethal, ethol, hexadecyl alcohol, or palmityl alcohol. The C18:0 fatty alcohol may be octadecanol, stearyl alcohol, 1-octadecanol, or octadecan-1-ol.

The recombinant Yarrowia lipolytica may further express a recombinant NADH kinase. The recombinant NADH kinase may comprise an amino acid sequence at least about 60%, 70%, 80%, 90%, 95%, 99% or 100% identical to the amino acid sequence of truncated Pos5 (SEQ ID NO: 8), also referred to as tyPos5. The recombinant NADH kinase may be expressed in the one or more peroxisomes of the recombinant Yarrowia lipolytica.

The recombinant Yarrowia lipolytica may overexpress one or more endogenous proteins. The one or more overexpressed endogenous proteins may be selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), triacylglycerol lipase (TGL4) (SEQ ID NO: 14), and a combination thereof. The endogenous protein may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous protein in a control Yarrowia lipolytica.

The recombinant Yarrowia lipolytica may overexpress endogenous isocitrate dehydrogenase enzyme (IDP3). The endogenous IDP3 may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous IDP3 in a control Yarrowia lipolytica.

The recombinant Yarrowia lipolytica may overexpress endogenous ADP/ATP translocase 1 (ANT1). The endogenous ANT1 may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous ANT1 in a control Yarrowia lipolytica.

The recombinant Yarrowia lipolytica may overexpress endogenous peroxisome biogenesis factor 11 (PEX11). The endogenous PEX11 may be overexpressed at a level at least about 10%, 20%, 30%, 40%, 50, 60%, 70%, 80%, 90% or 100%, or about 2, 3, 4, 5, 6, 7, 8, 9, 10, 100 or 1,000 times more than the expression level of the endogenous PE X11 in a control Yarrowia lipolytica.

The recombinant Yarrowia lipolytica may overexpress endogenous peroxisome biogenesis factor 16 (PEX1E). The endogenous PEX16 may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous PEX16 in a control Yarrowia lipolytica.

The recombinant Yarrowia lipolytica may overexpress endogenous ATP citrate lyase (ACL). The endogenous ACL may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous ACL in a control Yarrowia lipolytica.

The recombinant Yarrowia lipolytica may overexpress endogenous triacylglycerol lipase (TGL4). The endogenous TGL4 may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous TGL4 in a control Yarrowia lipolytica.

A method for producing one or more fatty alcohols is also provided. The production method comprises growing a recombinant Yarrowia lipolytica in a culture medium. The recombinant Yarrowia lipolytica comprises a heterologous polynucleotide encoding a fusion protein, and the fusion protein comprises a first amino acid sequence and a second amino acid sequence. The production method also comprises expressing the fusion protein in one or more peroxisomes in the recombinant Yarrowia lipolytica, and generating one or more fatty alcohols by the recombinant Yarrowia lipolytica. As a result, the one or more fatty alcohols are produced in the culture medium.

According to the production method, the first amino acid sequence may be at least about 60%, 70%, 80%, 90%, 95%, 99% or 100% identical to the amino acid sequence of 3-ketoacyl CoA thiolase (3KAT) (SEQ ID NO: 1), The first amino acid sequence may be the amino acid sequence of 3KAT. The second amino acid sequence may be at least about 60%, 70%, 80%, 90%, 95%, 99% or 100% identical to the amino acid sequence of a fatty acyl-CoA reductase (FAR), The FAR is an exogenous protein to Yarrowia lipolytica. The FAR may be TaFAR (SEQ ID NO: 2), MaACR (SEQ ID NO: 3) or MmFAR (SEQ ID NO: 4), The second amino acid sequence may be at least about 60%, 70%, 80%, 90%, 95%, 99% or 100% identical to the amino acid sequence of TaFAR, MaACR or MmFAR. For example, the second amino acid sequence may be the amino acid sequence of TaFAR, MaACR or MmFAR.

According to the production method, the first amino acid sequence may be at the N-terminus or C-terminus of the second amino acid sequence in the fusion protein. The fusion protein may comprise 3KAT and a FAR, wherein the 3KAT may be at the N-terminus or C-terminus of the FAR. The fusion protein may consist of 3KAT and a FAR, wherein the 3KAT may be at the N-terminus or C-terminus of the FAR. The FAR may be TaFAR, MaACR or MmFAR.

According to the production method, the fusion protein may further comprise a linker between the first amino acid sequence and the second amino acid sequence. The first amino acid sequence may be at the N-terminus of the linker, and the linker may be at the N-terminus of the second amino acid sequence. The first amino acid sequence may be at the C-terminus of the linker, and the linker may be at the C-terminus of the second amino acid sequence. The linker may comprise about 5-25 amino acids. The linker may consist of GGGGSGGGGSGGGGS (SEQ ID NO: 5), GGGGS (SEQ ID NO: 17), GGGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

According to the production method, the fusion protein may comprise 3KAT, a FAR and a linker, wherein the 3KAT may be at the N-terminus of the linker and the linker may be at the N-terminus of the FAR. The fusion protein may comprise 3KAT, a FAR and a linker, wherein the 3KAT may be at the C-terminus of the linker and the linker may be at the C-terminus of the FAR. The FAR may be TaFAR, MaACR or MmFAR. The linker may consist of GGGGSGGGGSGGGGS (SEQ ID NO: 5), GGGGS (SEQ ID NO: 17), GGGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

According to the production method, the fusion protein may consist of 3KAT, a FAR and a linker, wherein the 3KAT may be at the N-terminus of the linker and the linker may be at the N-terminus of the FAR. The fusion protein may consist of 3KAT, a FAR and a linker, wherein the 3KAT may be at the C-terminus of the linker and the linker may be at the C-terminus of the FAR. The FAR may be TaFAR, MaACR or MmFAR, The linker may consist of GGGGSGGGGSGGGGS (SEQ ID NO: 5), GGGGS (SEQ ID NO: 17), GGGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

According to the production method, the recombinant Yarrowia lipolytica may have knockout of one or more endogenous proteins. The one or more knocked-out endogenous proteins may comprise fatty acyl-CoA synthetase (FAA1) (SEQ ID NO: 15), fatty alcohol oxidase (FAO1) (SEQ ID NO: 16), or a combination thereof.

The production method may further comprise expressing a recombinant NADH kinase by the recombinant Yarrowia lipolytica. The recombinant NADH kinase may comprise an amino acid sequence at least about 60%, 70%, 80%, 90%, 95%, 99% or 100% identical to the amino acid sequence of truncated Pos5 (SEQ ID NO: 8). The recombinant NADH kinase may be expressed in the one or more peroxisomes in the recombinant Yarrowia lipolytica.

The production method may further comprise overexpressing one or more endogenous proteins by the recombinant Yarrowia lipolytica. The one or more overexpressed endogenous proteins may be selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), triacylglycerol lipase (TGL4) (SEQ ID NO: 14), and a combination thereof. The endogenous protein may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous protein in a control Yarrowia lipolytica.

The production method may further comprise overexpressing an endogenous protein, for example, isocitrate dehydrogenase enzyme (IDP3), ADP/ATP translocase 1 (ANT1), peroxisome biogenesis factor 11 (PEX11), peroxisome biogenesis factor 16 (PEXIG), ATP citrate lyase (ACL), or triacylglycerol lipase (TGL4), by the recombinant Yarrowia lipolytica. The endogenous protein may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous protein in a control Yarrowia lipolytica.

The production method may further comprise adding a solvent, for example, dodecane, to the culture medium to form a separate solvent layer above the culture medium before the growing. The resulting culture medium becomes biphasic.

According to the production method, the recombinant Yarrowia lipolytica may be grown in a continuous culture or fed-batch fermentation, in a continuous culture or fermentation, nutrients are added and targeted products are removed continuously. In a fed-batch fermentation, nutrients are added at different time points for an extended duration. A continuous culture or fermentation may be a better scaling up model for large scale industries to extract a product from the culture before it becomes toxic to the cells in the culture.

According to the production method, the culture medium may comprise the one or more fatty alcohols at a concentration of about 0.1-10, 0.1-5, 0.1-4, 0,1-3, 0.1-2, 0.1-1, 0.5-10, 0.5-5, 0.5-4, 0.5-3, 0,5-2, 0.5-1, 1-10, 1-5, 1-4, 1-3 or 1-2 gram per liter of the culture medium. The one or more fatty alcohols may comprise a C8:0 fatty alcohol, C10:0 fatty alcohol, C12:0 fatty alcohol, C16:0 fatty alcohol, C18:0 fatty alcohol, C18:1 fatty alcohol, or combination thereof. About 50-99%, 60-99%, 70-99%, 75-99%, 80-99%, 85-99%, 90-99%, 95-99%, 50-95%, 60-95%, 70-95%, 75-95%, 80-95%, 85-95%, 90-95%, 50-90%, 60-90%, 70-90%, 75-90%, 80-90%, 85-90%, 50-85%, 60-85%, 70-85%, 75-85%, 80-85%, 50-75%, 60-75% or 70-75% of the one or more fatty alcohols may be a C16:0 fatty alcohol. About 50-99%, 60-99%, 70-99%, 75-99%, 80-99%6, 85-99%, 90-99%, 95-99%, 50-95%, 60-95%, 70-95%, 75-95%, 80-95%, 85-95%, 90-95%, 50-90%, 60-90%, 70-90% , 75-90%, 80-90%, 85-90%, 50-85%, 60-85%, 70-85%, 75-85%, 80-85%, 50-75%, 60-75% or 70-75% of the one or more fatty alcohols may be a C18:0 fatty alcohol. The C16:0 fatty alcohol may be hexadecanol, cetanol, cetyl alcohol, ethal, ethol, hexadecyl alcohol, or palmityl alcohol. The C18:0 fatty alcohol may be octadecanol, stearyl alcohol, 1-octadecanol, or octadecan-1-ol.

A method for preparing a recombinant Yarrowia lipolytica is further provided. The preparation method comprises introducing a heterologous polynucleotide into Yarrowia lipolytica. The heterologous polynucleotide encodes a fusion protein. The fusion protein comprises a first amino acid sequence and a second amino acid sequence.

According to the preparation method, the first amino acid sequence may be at least about 60%, 70%, 80%, 90%, 95%, 99% or 100% identical to the amino acid sequence of 3-ketoacyl CoA thiolase (3KAT) (SEQ ID NO: 1). The first amino acid sequence may be the amino acid sequence of 3KAT. The second amino acid sequence may be at least about 60%, 70%, 80%, 90%, 95%, 99% or 100% identical to the amino acid sequence of a fatty acyl-CoA reductase (FAR), The FAR is an exogenous protein to Yarrowia lipolytica. The FAR may be TaFAR (SEQ ID NO: 2), MaACR (SEQ ID NO: 3) or MmFAR (SEQ ID NO: 4), The second amino acid sequence may be at least about 60%, 70%, 80%, 90%, 95%, 99% or 100% identical to the amino acid sequence of TaFAR, MaACR or MmFAR, For example, the second amino acid sequence may be the amino acid sequence of TaFAR, MaACR or MmFAR.

According to the preparation method, the first amino acid sequence may be at the N-terminus or C-terminus of the second amino acid sequence in the fusion protein. The fusion protein may comprise 3KAT and a FAR, wherein the 3KAT may be at the N-terminus or C-terminus of the FAR. The fusion protein may consist of 3KAT and a FAR, wherein the 3KAT may be at the N-terminus or C-terminus of the FAR. The FAR may be TaFAR, MaACR or MmFAR.

According to the preparation method, the fusion protein may further comprise a linker between the first amino acid sequence and the second amino acid sequence. The first amino acid sequence may be at the N-terminus of the linker, and the linker may be at the N-terminus of the second amino acid sequence. The first amino acid sequence may be at the C-terminus of the linker, and the linker may be at the C-terminus of the second amino acid sequence. The linker may comprise about 5-25 amino acids. The linker may be flexible, for example, consisting of the amino acid sequence of GGGGSGGGGSGGGGS (SEQ ID NO: 5), GGGGS (SEQ ID NO: 17), GGGGGGGG (SEQ ID NO: 18), or GGGSGGGSGGCSGOGS (SEQ ID NO: 19). The linker may be rigid, for example, consisting of the amino acid sequence of GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22) or (EAAAK), wherein n may be between 1 and 5, for example, EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26), and EAAAKEAAAKEAAAKEAAAKEAAAK (SEQ ID NO: 27).

According to the preparation method, the fusion protein may comprise 3KAT, a FAR and a linker, wherein the 3KAT may be at the N-terminus of the linker and the linker may be at the N-terminus of the FAR. The fusion protein may comprise 3KAT, a FAR and a linker, wherein the 3KAT may be at the C-terminus of the linker and the linker may be at the C-terminus of the FAR, The FAR may be TaFAR, MaACR or MmFAR. The linker may consist of GGGGSGGGGSGGGGS (SEQ ID NO: 5), GGGS (SEQ ID NO: 17), GGGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKE-AAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

According to the preparation method, the fusion protein may consist of 3KAT, a FAR and a linker, wherein the 3KAT may be at the N-terminus of the linker and the linker may be at the N-terminus of the FAR. The fusion protein may comprise 3KAT, a FAR and a linker, wherein the 3KAT may be at the C-terminus of the linker and the linker may be at the C-terminus of the FAR. The FAR may be TaFAR, MaACR or MmFAR. The linker may consist of GGGGSGGGGSGGGGS (SEQ ID NO: 5), GGGGS (SEQ ID NO: 17), GGGGGGGG (SEQ ID NO: 18), GGGSGGGSGGGSGGGS (SEQ ID NO: 19), GSAGSAAGSGEF (SEQ ID NO: 6), AEAAAKEAAAKEAAAKEAAAKALEAEAAAKEAAAKEAAAKEAAAKA (SEQ ID NO: 20), PAPAP (SEQ ID NO: 21), AEAAAKEAAAKA (SEQ ID NO: 22), EAAAK (SEQ ID NO: 23), EAAAKEAAAK (SEQ ID NO: 24), EAAAKEAAAKEAAAK (SEQ ID NO: 25), EAAAKEAAAKEAAAKEAAAK (SEQ ID NO: 26) or EAAAKEAAAKEAAAKEAAAKEAAAK (SEQ ID NO: 27).

The preparation method may further comprise knocking out one or more endogenous proteins. The one or more knocked-out endogenous proteins may be selected from the group consisting of fatty acyl-CoA synthetase (FAA1) (SEQ ID NO: 15), fatty alcohol oxidase (FAO1) (SEQ ID NO: 16), or a combination thereof.

According to the preparation method, the recombinant Yarrowia lipolytica may express the fusion protein in one or more peroxisomes in the recombinant Yarrowia lipolytica and generate one or more fatty alcohols. The one or more fatty alcohols may be generated in the one or more peroxisomes. The one or more fatty alcohols may be secreted by the recombinant Yarrowia lipolytica. The one or more fatty alcohols may a C8:0 fatty alcohol, C10:0 fatty alcohol, C12:0 fatty alcohol, C16:0 fatty alcohol, C18:0 fatty alcohol, C18:1 fatty alcohol, or combination thereof. The C16:0 fatty alcohol may be hexadecanol, cetanol, cetyl alcohol, ethal, ethol, hexadecyl alcohol, or palmityl alcohol. The C18:0 fatty alcohol may be octadecanol, stearyl alcohol, 1-octadecanol, or octadecan-1-o.

According to the preparation method, the recombinant Yarrowia lipolytica may express the fusion protein and a recombinant NADH kinase. The recombinant NADH kinase may comprise an amino acid sequence at least about 60%, 70%, 80%, 90%, 95%, 99% or 100% identical to the amino acid sequence of truncated Pos5 (SEQ ID NO: 8). The recombinant NADH kinase may be expressed in the one or more peroxisomes in the recombinant Yarrowia lipolytica.

According to the preparation method, the recombinant Yarrowia lipolytica may express the fusion protein and overexpress one or more endogenous proteins. The one or more overexpressed endogenous proteins may be selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), triacylglycerol lipase (TGL4) (SEQ ID NO: 14), and a combination thereof. The endogenous protein may be overexpressed at a level at least about 10%, 20%, 30%, 40%, 50%, 60%, 70%, 80%, 90% or 100%, or about 2, 3, 4, 5, 6, 7, 8, 9, 10, 100 or 1,000 times more than the expression level of the endogenous protein in a control Yarrowia lipolytica.

For each preparation method of the present invention, a recombinant Yarrowia lipolytica prepared by the method is provided.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Harnessing peroxisomes for fatty alcohol production in Yarrowia lipolytica

### A, Methods and Materials

### 1. Plasmid Construction

All plasmids used in this study were constructed using T4 ligation and SLIC assembly methods. Leu and/or URA3 based plasmids/cassettes were selected to construct the genes in recombinant Y. lipolytica. Table 1 provides the amino acid sequences of some proteins expressed or knocked out in the recombinant Y, lipolytica.

Construction of episomal expression cassettes: The following genes IDP3, tyPos5, PEX11, PEX16, ACL, TGL4 were PCR-amplified from Y. lipolytica PO1f gDNA and inserted into expression vector URA3 background plasmid p1W245 and Leu background plasmid pSL16, both contains UAS1B8-TEF(136)-hrGFP-CYCIt. All the genes were cloned into the respective episomal expression vectors using AscI/NheI digestion to replace brGFP with genes using T4 DNA ligase and enables constitutive expression in Y. lipolytica. All the episomal expression plasmids constructed in this study are listed in the Table 2 and primers used to construct plasmids are listed in Table 3.

Construction of target genome integrative expression cassettes. CRISPR-Cas9 markerless integration Leu background plasmids pCRISPRyl_D17 and pCRISPRyl_AXP were used in this study for targeting the genomic loci in the Y. lipolytica strain. The following genes TaFAR, MaACR, MmFAR were codon optimized for Y. lipolytica expression and synthesized by Genscript (Table 5). The genes were PCR amplified and fused with 3KAT gene using overlap extension PCR. All fusion gene constructs were cloned into the homology donor URA3 background plasmids pHR_D17_hrGFP and pHR_AXP_hrGFP using AscI/NheI digestion and T4 DNA ligase for ligation. Target genome integrative plasmids cassette constructed in this study are listed in the Table 2. These co-expression plasmids enabled integrative selection with leucine and uracil auxotrophy and expression of enzymes at the target loci in the genome. Further, subsequent curing procedure has removed both leucine and uracil markers present in the cells. CRISPR-Cas9 based knockout plasmids pCas9-Leu and pCas9-Ura were used to construct the double cut knockout system. Knockout plasmids were generated using sequence and ligation independent cloning (SLIC) by insertion of two annealed oligonucleotides containing gene target sequences of gRNAs and SLIC overhangs into parent plasmid digested with NsiI. The knockout plasmids constructed in this study are presented in Table 2.

2, Method used to construct engineered Y. lipolytica strains

All constructed episomal expression vectors or markerless genome targeted integration plasmids were transformed into Y. lipolytica POIf cells using the lithium acetate method. Y. lipolytica strains was plated on solid YPD medium and incubated at 30° C. for 36 hours. Fresh one-step buffer was prepared as follows: 90 μL of 50% PEG 4000 (sterile Filtered), 5 μL of 2 M lithium acetate (sterile filtered), 2.5 μL of salmon sperm DNA (ssDNA was boiled at 98˜C for 10 minutes) and 2.5 μl of 2M dithiothreitol (DTT). A loop full of Y. lipolytica was taken and mixed with the one-step buffer. Then, 250-500 ng of plasmid DNA was then added in tube contains one-step buffer and cells, which then mixed and vortexed for 10 seconds. The tubes were then incubated at 39° C. water bath for 1 hour to facilitate the transformation of plasmids into the cells. After incubation, 100 μl of the cell mixture was plated on its respective YSC selective plates and incubated at 30° C. for 2 days.

Multiple colonies obtained from the YSC selection plates were screening using colony PCR targeting the insert with gene specific primers sets. In case of genome integration strain engineering, colonies exhibit positive integration of genes were cured for the two plasmids marker system by overnight growth in rich media YPD supplemented with 5-fluoroorotic acid (5-FOA). Final engineered markerless strains was confirmed by PCR fragment size with gene specific primers and subsequently by Sanger sequencing. All the strain constructed in this study was listed in the Table 4.

### 3. Visualization of Enzyme Localization Using Fluorescence Microscopy

To confirm 3KAT enzyme is effectively localizing TaFAR into the peroxisomes, Green Fluorescent Protein (GFP) was fused at the C-terminus of 3KAT using the flexible linker (GGGS)3 and then the cassettes were transformed into the Y. lipolytica strain. Cells expressing GFP in the cytosolic space was used as a control strain for non-localization target. Both strains were grown for 48 h in YSC media containing 20 g/L glucose at 30° C. and 220 rpm, GFP localization was assessed using a Zeiss LSM 880 confocal microscope with a 63×/1.4 objective. GFP was excited at 488 nm and its fluorescence collected above 500 nm.

### 4. Flask Fermentation for Biphasic Cultivation of Fatty Alcohol

In this study, Escherichia coli DH5a cells were used for plasmid construction and cultured in LB (Luria-Bertani) medium at 37° C. in 250 rpm with 100 mg/L ampicillin to maintain the plasmids. YPD media was used to generate frozen stocks and preparing cells for transformation which contains 10 g/L yeast extract, 20 g/L peptone and 20 g/L glucose. Engineering Y. lipolytica strains tube fermentation for preculture was performed in 14 mL culture tubes containing 2 mL media incubated at 30° C. and 220 rpm. Y. lipolytica cells were inoculated from 48 h precultures to shake flasks cultivation at an initial cell density of 0.2 at 600 nm (0D600) at 220 rpm and 30° C. for 7 days of flask fermentation. All engineered strains for fatty alcohol production were cultivated in 125 mL baffled flasks contains 12 mL defined YSC drop out media containing 6.7 g/L Yeast Nitrogen Base (without ammonium sulfate), 1,365 g/L ammonium sulfate and 0.69 g/L of CSM-leu and/or CSM-ura in 80 g/L of glucose as sole carbon source.

Continuous extractive fermentation was performed in flask cultivation to extract the fatty alcohols production from engineered strains by adding dodecane (Sigma) as overlay. The impact of dodecane in cell growth was initially assessed by addition of dodecane at different time points in cell growth. To minimize the impact of dodecane in the growth of engineering Y. lipolytica strain, we added 3 mL of dodecane overylay in 12 mL of cells at 36 h for the continuous extraction of fatty alcohols and maintained as biphasic culture as aqueous: organic emulsion throughout the flask fermentation,

### 5. Fed-Batch Fermentation in Bioreactor for Fatty Alcohol Production

Based on the flask experiment result, the best Fatty alcohol production strain FS113 was scaled up in a 5 L Eppendorf BioFlo 320 with 10% of dodecane extractive layer. The initial cell density was 0.15 OD600 was used with a total operating volume of 2 L with the media contained 80 g/L glucose, 6.7 g/L Yeast Nitrogen Base, 0.69 g/L CSM-Leu-Ura and 1,365 g/L ammonium sulfate. First glucose pulse of 80 g was added at approximately 72 h (Stock concentration of 600 g/L). In this study, a second glucose pulse of 80 g was added at approximately 144 h to prevent glucose exhaustion and to keep the cells at active lipogenesis phase. Dissolved oxygen was maintained at 40% using an agitator cascade and pH was controlled to 5.0 using 3M NaOH. The pH control (base addition) balanced evaporative loss maintaining the volume at 1.7 L throughout the fermentation. Air was sparged into the bioreactor at a rate of 0.2-2.5 splm. Fermentation was lasted for 10 days with samples collected daily for analysis. Sterile antifoam 204 (Sigma) was added as necessary during the run. Biomass accumulation was determined for 1 mL of cells by washing in PBS and completely evaporating any residual liquid at 90C for >1 h. All the bioreactor samples obtained at different time profile during the run (biomass, sugar, lipids, alcohols) were measured in duplicate.

### 6. Extraction & Quantification of Fatty Alcohols

500 μL of top overlay broth from the fermentation cultures was taken and centrifuged at 10,000×g for 10 min and carefully pipetted out the dodecane overlay to quantify the fatty alcohol concentration, Fatty alcohols concentration from dodecane overlay was analyzed by Trace 1310 Gas Chromatography (ThermoFisher Scientific) equipped with Flame Ionization Detection system (FID, ThermoFisher Scientific) and DB-WAXETR (Agilent) 30 m, 0.25 mm, 0.25 μm-JW GC column. The injected volume of sample was 1 μL (split ratio of 5) with the flow rate of 1 mL/min. The initial oven temperature was set at 50° C. and ramped to 150° C. at the ramp rate of 25° C./min and maintained for 2 min, followed ramping the oven temperature to final 250° C. at the ramp rate of 4.0° C./min to the total run time for 41 minutes,

### 7. Extraction and Quantification of Fatty Acids

To quantify the fatty acid concentration, 1 mL cells were collected from the fermentation and centrifuged for 4500×g for 5 min. Cell pellets were added with 100 mL of Internal standard (IS) solution (2 mg/L methyl pentadecanoate and 2 mg/L glyceryl triheptadecanoate) and resuspended with 500 uL 0.5N sodium methoxide (20 g/L sodium hydroxide in methanol). Cells were vortexed for 40 mins at 2000 rpm on heavy-duty vortex. 40 mL of concentrated sulfuric acid added directly on the tube. Further, 850 mL of hexane was added in the tube to neutralize the samples and vortexed for 2000 rpm for 20 mins at heavy-duty vortex. Finally, samples were centrifuged for 1 minute at maximum rpm to obtained the hexane layer containing Fatty acid methyl esters (FAME's). Samples were analyzed for FAME's at GC-FID in DB-23 (Agilent) 30 m, 0.25 mm, 0.25 um column. The GC program was as follows: Initial oven temperature of 50° C. and ramped to 175° C. at the ramp rate of 25° C./min and maintained for 1 min, followed ramping the oven temperature to 230° C. at the ramp rate of 4.0C/min and hold for 5 min, then final ramping to 250° C. at the ramp rate of 25° C. and hold it for 2 mins.

### 8. Extraction and Quantification of Glucose and Citrate

The glucose and citrate time profile in fed-batch bioreactor run was determined by high-performance liquid chromatography analysis. 1 mL broth sample was taken at different time point in the bioreactor run and centrifuged at 12,000×g for 10 min, filtered through a 0.2-μm syringe filter, and analyzed with an Aminex HPX-87H column (Bio-Rad) on a 1260 Infinity II HPLC (Agilent) which is equipped with Refractive Index Detector (RID). The column was eluted with 5 mM H₂SO₄ as solvent at a flow rate of mobile phase was 0.6 mL/min at 50° C. for 30 min. The standard curve for glucose and citrate was performed using the same procedure.

### B. Results

### 1. Compartmentalization by 3KAT Fusion Enhances Peroxisome Localized Fatty Alcohol Production

Biosynthetic production of metabolites in yeast is often limited by intermediate product related toxicity, and formation of byproducts by competing endogenous. To address this limitation, the inventors aimed to harness the subcellular organelles peroxisome for the fatty alcohol production. Initially, the base strain Y. lipolytica Po1f was constructed with ΔFAA, ΔFAO to avoid common fatty alcohol degradation pathways. Three different Fatty acyl reductase (FAR)'s enzymes, TaFAR, MaACR and MmFAR from Tyto alba, Marinobacter aquaeolei, and Mus musculus, respectively, were selected for this study.

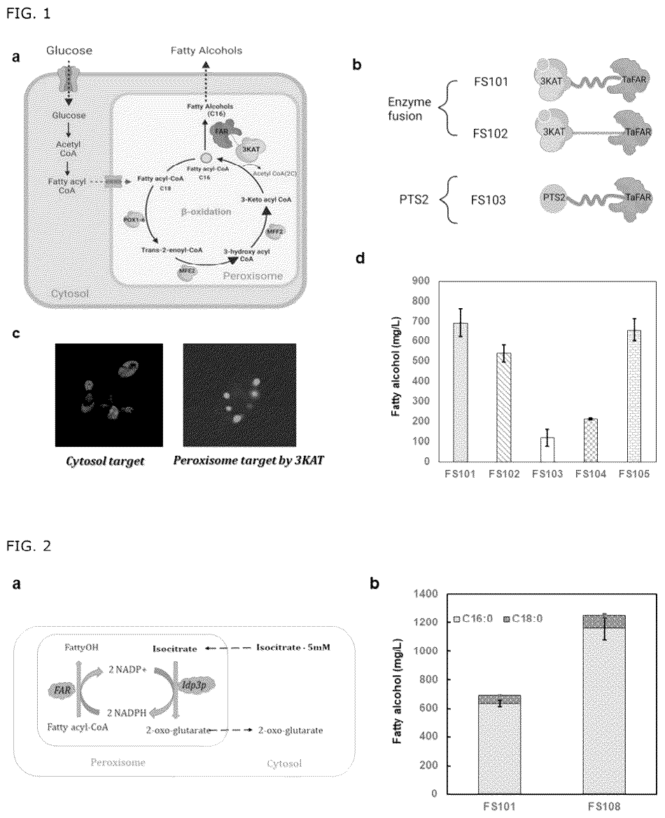

Next, the invents attempted to closely localize the fatty alcohol biosynthetic pathway with the beta oxidation pathway. It was hypothesized that localization of FARs with the beta oxidation pathway would give the FARs more access to acyl-CoA substrates, and improve fatty alcohol production. To achieve it, enzyme fusion complex constructed by physically positioning non-native enzyme FAR's (TaFAR, MaACR, MmFAR) in close proximity to 3-ketoacyl-CoA thiolase (3KAT, also known as POT1), a native enzyme present in peroxisomal beta-oxidation (FIG. 1A). Enzyme fusions were constructed with FAR's fused to the C-terminus of 3KAT, using either a flexible linker GGGGSGGGGSGGGGS (SEQ ID NO: 5) (FS101) or a rigid linker GSAGSAAGSGEF (SEQ ID NO: 6) (FS102) (FIG. 1b). The strain FS101 expressing the 3KAT/TaFAR fusion protein with flexible linker produced fatty alcohols at 693 mg/L, whereas the strain FS102 expressing the 3KAT/TaFAR fusion using rigid linker produced fatty alcohols at 475 mg/L: Likewise, the strain FS104 expressing the 3KAT/MaACR and the strain FS105 expressing the 3KAT/MmFAR fusion protein constructed using the flexible linker produced total fatty alcohols at 211.7 mg/L and 657 mg/L, respectively (FIG. 1d). Peroxisome localization efficiency of 3KAT was tested by fusing 3KAT with GFP as reporter gene (FS107) (FIG. 1c, right panel) and examined under confocal microscopy as compared with cytosolic expression of GFP (FS106) (FIG. 1c, left panel), The confocal images show clear localization of GFP in peroxisomes of Y. lipolytica using 3KAT as the peroxisome localization protein (FIG. 1c). WoLFPSORT protein localization predication tool was then used to identify the presence of native peroxisome target signal type 2 (PTS2) tag in 3KAT, a nine amino acid sequence long tag at the N-terminus, that naturally helps the enzyme to localize into the peroxisomes, To determine if the PTS2 tag alone was sufficient to explain the benefit of 3KAT fusion on fatty alcohol production in peroxisomes, strain FS103 in which PTS2 directly fused with N-terminus of TaFAR was constructed. The result shows the presence of PTS2 was detrimental to the alcohol production in peroxisomes (FIG. 1d), thus confirming that fusion to 3KAT responsible for the effect in increasing the compartmentalized production of alcohol in peroxisome.

### 2. Enhancing Auxiliary Enzyme Supply in the Peroxisome

Next, the inventors systematically identified the bottlenecks in the peroxisome to increase its capacity in production of fatty alcohols. Biosynthesis of fatty alcohol from fatty acyl-CoA carried out by two-step reduction, which requires 2 NADPH to drive the reduction reaction by fatty acyl-CoA reductase enzyme. The peroxisome contains an endogenous auxiliary enzyme isocitrate dehydrogenase (IDP3) to catalyze the reaction of isocitrate to a-ketoglutarate and reduced cofactor NADPH, which is essential to 2,4 dienoyl-CoA reductase enzyme for the degradation of polyunsaturated fatty acids with double bonds at even-numbered position (FIG. 2a). Initially to identify the limitation of redox power in peroxisome on alcohol production, exogenous addition of isocitrate was supplied directly in the culture and improvement in fatty alcohol titer was shown, Thus, strain FS108 was constructed to overexpress endogenous auxiliary enyne IDP3 in FS101 background to improve the NADPH pool in piroximone and achieved 1.25 g/L of fatty alcohols (FIG. 2b). IDP3 is the only known enzyme to produce NADPH in peroxisome of S. cerevisiae. To test if IDP3 is the only source of NADPH in Y. lipolytica, strain FS120 in which IDP3 was knocked out was constructed, and a growth challenge was performed on a polyunsaturated fatty acid as the sole carbon source. FS120 had severely impaired growth in linoleic acid (data not shown), proving that IDP3 is the only NADPH supplier present in peroxisome of Y. lipolytica, and the diffusion of NADPH across the membrane bound organelles was not observed.

### 3. Enhancing NADPH in the Peroxisome Increases Fatty Alcohol Production

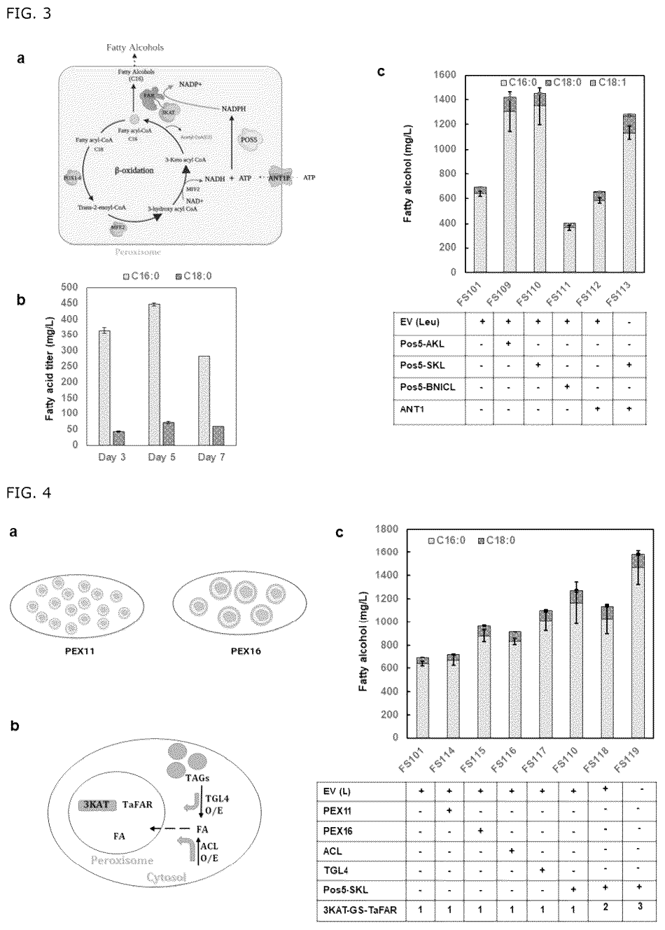

An alternative to increase the peroxisomal NADPH, we localized the NADH kinase from Y. lipolytica to the peroxisome to convert NADH derived from beta oxidation and ATP into NADPH. The POS5 from S. cerevisiae has a low Km value of 0.49 mM for NADH and localized in mitochondrial matrix. We identified a POS5 homolog (ylPos5) in the Y. lipolytica genome, which also contains a mitochondrial signal tag at its N-terminus. We truncated the mitochondrial localization signal to obtain a truncated POS5 (tyPos5) and fused with PTS1 tags AKL, SKL and BNICL at C-terminus of tyPos5 to construct the strains FS109, FS110 and FS111, respectively. The strains with peroxisome localized NADH kinase had increased fatty alcohol production, presumably by enhancing the co-factor supply specifically in the peroxisome (FIG. 3a). The strain FS110 has produced the highest fatty alcohol of 1.45 g/L after 7 days of batch flask culture (FIG. 3c). In addition, localization of NADH kinase also increased the secretion of free fatty acids such as palmitic acid (C16:0) and stearic acid (C18:0) in the media after 7 days of flask culture (FIG. 3b). Finally, to increase the production of compartmentalized production NADPH from ATP, we overexpressed the peroxisomal ATP transporter ANT1 individually and coexpressed with tyPos5 fused to SKL. The strain FS112 and FS113 has produced the new C18:1 distribution in fatty alcohol profile (FIG. 3c). However, the total fatty alcohol titer was not increased by ANT1 overexpression.

### Example 4. Peroxisome Biogenesis and Precursor Flux Improvement for Enhanced Fatty Alcohol Production

Peroxisome is a membrane-bound organelle that contains essential processes such as oxidation of fatty acids and detoxification of H₂O₂. PEX11 overexpression promotes the reduction in peroxisomes size and increase in peroxisomes number in strain FS114. Whereas, the strain FS115 overexpressing PEX16 leads to enlargement in peroxisomes size and decreases the peroxisome numbers (FIG. 4a). In our hands, the strain FS115 overexpressing PEX16 has increased the fatty alcohol titer to 996 mg/L compared to ES114 which had a modest decrease in titer. These data indicate larger peroxisomes are beneficial for peroxisomal fatty alcohol production.

Next, we increased the precursor for the fatty alcohol production by enhancing the accumulation of free fatty acid in cytosol. Firstly, we overexpressed the ATP citrate lyase (ACL) which catalyzes the reaction from citrate to generate cytosolic acetyl-CoA, that in turn leads to increased lipid flux. Secondly, triacylglycerol lipase (TGL4) was overexpressed to enable the lipolysis of stored triacylglycerol (TAG) and release free fatty acids in cytosol (FIG. 4b). We hypothesize the accumulation of the free fatty acid drives increased transport into the peroxisome for fatty alcohol production. The strain FS116 and FS117 have significantly improved both fatty acid and fatty alcohol production (FIG. 4c). In addition, we constructed multiply copies to increase the copy number of 3KAT fusion in Y. lipolytica to enhance the production of fatty alcohols. The strain FS119 contains 3 copies of 3KAT fused TaFAR and coexpressed with tyPos5 fused to SKL had only little impact on fatty alcohol production (FIG. 4c).

### Example 5. Fed-Batch Fermentation for Fatty Alcohol Production from Bioreactor

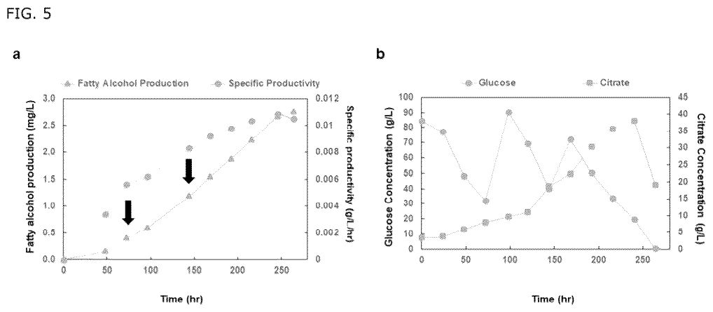

Finally, we demonstrated the capacity of peroxisome organelles engineered Y. lipolytica for the compartmentalized production of fatty alcohols from flask culture to 2 L bioreactor in Eppendorf Bioflo320, We tested the strain ES110 for fatty alcohol production in fed-batch fermentation for 260 h with controlled pH and dissolved oxygen (DO). The target pH was about 5±0,1, The target DO was about 40%. To keep the cells in active lipogenesis and prevent exhaustion of glucose, we spiked two rounds of 80 g glucose for every 3 days of fermentation at 72 h and 144 h, Result shows the fatty alcohol production was improved in bioreactor to 2.77 g/L of total fatty alcohol of which the 91% hexadecanol (C16:0) and 9% of octadecanol (C18:0) (FIG. 5a). Based on the previous literature survey, we report our subcellular organelle engineered strain has achieved the highest production of C16:0 hexadecanol of 2.53 g/L (Table 6). Glucose utilization was measured throughout the culture. Citrate was steadily accumulated throughout the growth of the culture in glucose, then it consumed after glucose get depleted in the fermentation run at 260 h of fermenter (FIG. 5b).

TABLE 1
Amino acid sequences

| | | | SEQ |
|---|---|---|---|
| Name | Organism | Sequence | ID NO |
| 3KAT | Yarrowia | MDRLNNLATQLEQNPAKGLDAITSKNPDDVVITAAYR | 1 |
| | lipolytica | TAHTKGGKGLFKDTSSSELLASLLEGLVKESKIDPKLI | |
| | | GDVVCGNVLAAGAGATEHRAACLVAGIPETVPFVALN | |
| | | RQCSSGLMAVNDVANKIRAGQIDIGIGCGVESMSNQ | |
| | | YGPNSVTPFSNKFQNNEEAKKCLIPMGITSENVAAKY | |
| | | NVSRKAQDAFAAKSYEKAAAAQAAGKFDQEILPIKTT | |
| | | VLDDDDNEKEVTVNKDDGIRPGVTAEKLGKLKPAFSA | |
| | | EGTTHAGNASQISDGAGAVLLMRRSVAEKLGQPILAK | |
| | | FVHCKTVGVPPELMGIGPAYAIPAVLEDLGLTVNDVDV | |
| | | FEINEAFASQALFSIQHCGIDESKVNPRGGAIAIGHPL | |
| | | GATGARQFATLLSELKESGKKVGVTSMCIGTGMGAAS | |
| | | LVVAE | |
| TaFAR | Tyto alba | MVSIPEYYEGKNILLTGATGFMGKVLLEKLLRSCPKVK | 2 |
| | | AVYVLVRHKAGQTPEARIEEITNCKLFDRLRDEQPDFK | |
| | | AKTIVITSELTQPELDLSEPIKEKLIERINIIFHCAATVRF | |
| | | NETLRDAVQLNVTATQQLLFLAQRMKNLEVFMHVSTA | |
| | | YAYCNRKQIEEIVYPPPVDPKKLIDSLEWMDDGLVNDI | |
| | | TPKLIGDRPNTYTYTKALAEYVVQQEGAKLNTAIIRPSI | |
| | | VGASWKEPFPGWIDNFNGPSGLFIAAGKGILRTMRAS | |
| | | NSAVADLVPVDVVVNTTLAAAWYSGVNRPRNVMIYN | |
| | | CTTGGTNPFHWGEVGYHINLNFKINPLENAVRHPNCS | |
| | | LQSNPLLHQYWTAVSHTMPAFLLDLLLRLTGHKPWMM | |
| | | KTITRLHKAMMLLEYFTSNSWIWNTENMTMLMNQLN | |
| | | PEDKKTFNFDVRQLHWAEYMENYCMGTKKYVLNEEM | |
| | | SGLPAARKHLNKLRNIRYGFNTVLVILIWRIFIARSQM | |
| | | ARNIWYFVVSLCYKFLSYFRASSTMRY | |
| MaACR | Marinobacter | MAIQQVHHADTSSSKVLGQLRGKRVLITGTTGFLGKV | 3 |
| | aquaeolei | VLERLIRAVPDIGAIYLLIRGNKRHPDARSRFLEEIATS | |
| | | SVFDRLREADSEGFDAFLEERIHCVTGEVTEAGFGIGQ | |
| | | EDYRKLATELDAVINSAASVNFREELDKALAINTLCLR | |
| | | NIAGMVDLNPKLAVLQVSTCYVNGMNSGQVTESVIKP | |
| | | AGEAVPRSPDGFYEIEELVRLLQDKIEDVQARYSGKVL | |
| | | ERKLVDLGIREANRYGWSDTYTFTKWLGEQLLMKALN | |
| | | GRTLTILRPSIIESALEEPAPGWIEGVKVADAIILAYARE | |
| | | KVTLFPGKRSGIIDVIPVDLVANSIILSLAEALGEPGRR | |
| | | RIYQCCSGGGNPISLGEFIDHLMAESKANYAAYDHLFY | |
| | | RQPSKPFLAVNRALFDLVISGVRLPLSLTDRVLKLLGN | |
| | | SRDLKMLRNLDTTQSLATIFGFYTAPDYIFRNDELMAL | |
| | | ANRMGEVDKGLFPVDARLIDWELYLRKIHLAGLNRYA | |
| | | LKERKVYSLKTARQRKKAAY | |
| MmFAR | Mus | MVSIPEYYEGKNILLTGATGFLGKVLLEKLLRSCPRVNS | 4 |
| | musculus | VYVLVRQKAGQTPQERVEEILSSKLFDRLRDENPDFRE | |
| | | KIIAINSELTQPKLALSEEDKEIIIDSTNVIFHCAATVRF | |
| | | NENLRDAVQLNVIATRQLILLAQQMKNLEVFMHVSTA | |
| | | YAYCNRKHIDEVVYPPPVDPKKLIDSLEWMDDGLVND | |
| | | ITPKLIGDRPNTYIYTKALAEYVVQQEGAKLNVAIVRPS | |
| | | IVGASWKEPFPGWIDNFNGPSGLFIAAGKGILRTMRA | |
| | | SNNALADLVPVDVVVNTSLAAAWYSGVNRPRNIMVY | |
| | | NCTTGSTNPFHWGEVEYHVISTFKRNPLEQAFRRPNV | |
| | | NLTSNHLLYHYWIAVSHKAPAFLYDIYLRMTGRSPRM | |
| | | MKTITRLHKAMVFLEYFTSNSWVWNTDNVNMLMNQL | |
| | | NPEDKKTFNIDVRQLHWAEYIENYCMGTKKYVLNEEM | |
| | | SGLPAARKHLNKLRNIRYGENTILVILIWRIFIARSQMA | |
| | | RNIWYFVVSLCYKFLSYFRASSTMRY | |
| Linker | Synthetic | GGGGSGGGGSGGGGS | 5 |
| | Construct | | |
| Linker | Synthetic | GSAGSAAGSGEF | 6 |
| | Construct | | |
| Pos5 | Yarrowia | MRLLIRRTGLTRPHGVQARRSTWIRLLSTEILHAELLP | 7 |
| | lipolytica | DRQSPHYVQESTSLSSLVWDKPLENVLIVKKPWDHN | |
| | | VRESLIQMASHIQRRYPRVNILVEEHVADEVQKQIGA | |
| | | AGVTAIHTGPGEVLRNKTDLLVTLGGDGTILHATSMFA | |
| | | SGEVPPVLSFSLGTLGFLLPFDFKDFKTAFDMVYSSQA | |
| | | SVVNRARLACQKMSIRKEITHLPSQSHIEHNSTHVYG | |
| | | NPDDYNLSPLTYAMNDINIHRGAEPHLTKLDIHVDGEF | |
| | | ITRAIADGVTIATPTGSTAYSLSSGGSIVHPRVACILLT | |
| | | PICPRSLSFRPLIFPATSKICITASSESRGRGAELSVDG | |
| | | IAKGLVRPSDKILVESETGHNSGIWCVAKTDRDWVS | |
| | | GLNGLLGFNSSFGKGGEASGDVA | |
| Truncated | Synthetic | MELLPDRQSPHYVQESTSLSSLVWDKPLENVLIVKKP | 8 |
| Pos5 or | construct | WOHNVRESLIQMASHIQRRYPRVNILVEEHVADEVQK | |
| tyPos5 | derived from | QIGAAGVTAIHTGPGEVLRNKTDLLVTLGGDGTILHAT | |
| | full length | SMFASGEVPPVLSFSLGTLGFLLPFDFKDFKTAFDMVY | |
| | sequence of | SSQASVVNRARLACQKMSIRKEITHLPSQSHIEHNST | |
| | Yarrowia | HVYGNPDDYNLSPLTYAMNDINIHRGAEPHLTKLDIHV | |
| | lipolytica | DGEFITRAIADGVTIATPTGSTAYSLSSGGSIVHPRVA | |
| | | CILLTPICPRSLSFRPLIFPATSKICITASSESRGRGAEL | |
| | | SVDGIAKGLVRPSDKILVESETGHNSGIWCVAKTDRD | |
| | | WVSGLNGLLGFNSSFGKGGEASGDVA | |
| IDP3 | Yarrowia | MLTPATTRLLALSKTMSTTATRGLSTKIKVKNPIVELD | 9 |
| | lipolytica | GDEMTRIIWKSIKDKLILPYLDIDLKYYDLGIEYRDQTN | |
| | | DQVTIDAAEAIKKYQVGVKCATITPDEARVKEFGLKK | |
| | | MWLSPNGTIRNILGGTVFREPIVIPAVPRLVPGWKEPII | |
| | | IGRHAHGDQYKAQDAVIPGAGELTLNFKPANGGDEQ | |
| | | VIKVYTYDAPGVAMAMYNTDESITGFAYSSFNLALQK | |
| | | KLPLYMSTKNTILKKYDGRFKDIFQEIYDKEYKDKFDA | |
| | | AGIWYEHRLIDDMVAQMIKSKGGFIMALKNYDGDVQ | |
| | | SDIVAQGFGSLGLMTSVLVTPDGKTFESEAAHGTVTR | |
| | | HYRQHQQGKETSTNSIASIFAWTRGLIQRGILDETPE | |
| | | VTKFAEALEKATVDTVDKDGIMTKDLALAGGKTDRSS | |
| | | YVLTEEFIDAVANRLKKDLA | |
| ANT1 | Yarrowia | MAAISKDYVLSPWGKAVAGAAGAVLANTLVYPLDIVK | 10 |
| | lipolytica | TRLQVQVKRKEGGPLPAFEEGHFEHYEGTVDALKKIYA | |
| | | ANGLAGLYQGLPSCLLGVASTNFAYFYWYGFIRDSYIK | |
| | | RNPGKALSTPIELLLGAVAGALAQVFTIPVAVITTRQQT | |
| | | SDAKSRQGFLATAKSVVDDDGISGLWRGLKASLVLVI | |
| | | NPSITYGSFERLRTILFKGKLHLSPGENFLLGALSKAMA | |
| | | TIATQPMIVAKVMQQSKTKGGKQFNSFVQALVFLFKE | |
| | | EGILGMWKGVGPQISKGIIVQGLLFMIKDQVELFIVLL | |
| | | FRLMKAPTLIKG | |
| PEX11 | Yarrowia | MVSKLVGRTKPAGDSQKTVRIDEKVEIKEFQAHKPEII | 11 |
| | lipolytica | DNTTEIPAPVPMSIAERSIADAARPPLHASPWRVFLTLF | |
| | | AEKGGLDKTIKLIQYTGRLLLWAAKQGWFTRHKQMM | |
| | | LLWKLAEMESRLNGMVSNFSQFRKIIKLGEWLGPVED | |
| | | LVTTKNPLTSLAFQSELMEVINTIGDDIYCLSKIGVVKG | |
| | | KRLGRNGELMANWGWYGAIFINIKVGIETYQLAAKSG | |
| | | DEAAIWDAKLTLFKLANDFIFCTIDCLEPEGLSNIYQTV | |
| | | TGLASGSVGFYKLWRKISKKLDKELEEKERCKTC | |
| PEX16 | Yarrowia | MTDKLVKVMQKKKSAPQTWLDSYDKFLVRNAASIGS | 12 |
| | lipolytica | IESTLRTVSYVLPGRENDVEIATETLYAVLNVLGLYHDT | |
| | | IIARAVAASPNAAAVYRPSPHNRYTDWFIKNRKGYKY | |
| | | ASRAVTFVKFGELVAEMVAKKNGGEMARWKCIIGIEG | |
| | | IKAGLRIYMLGSTLYQPLCTTPYPDREVTGELLETICRD | |
| | | EGELDIEKGLMDPQWKMPRTGRTIPEIAPTNVEGYLLT | |
| | | KVLRSEDVDRPYNLLSRLDNWGVVAELLSILRPLIYAC | |
| | | LLFRQHVNKTVPASTKSKFPFLNSPWAPWIIGLVIEAL | |
| | | SRKMMGSWLLRQRQSGKTPTALDQMEVKGRTNLLG | |
| | | WWLFRGEFYQAYTRPLLYSIVARLEKIPGLGLFGALIS | |
| | | DYLYLFDRYYFTASTL | |
| ACL | Yarrowia | MSANENISRFDAPVGKEHPAYELFHNHTRSFVYGLQP | 13 |
| | lipolytica | RACQGMLDFDFICKRENPSVAGVIYPFGGQFVTKMYW | |
| | | GTKETLLPVYQQVEKAAAKHPEVDVVVNFASSRSVYS | |
| | | STMELLEYPQFRTIAIIAEGVPERRAREILHKAQKKGVT | |
| | | IIGPATVGGIKPGCFKVGNTGGMMDNIVASKLYRPGS | |
| | | VAYVSKSGGMSNELNNIISHTTDGVYEGIAIGGDRYP | |
| | | GTTFIDHILRYEADPKCKIIVLLGEVGGVEEYRVIEAVK | |
| | | NGQIKKPIVAWAIGTCASMFKTEVQFGHAGSMANSD | |
| | | LETAKAKNAAMKSAGFYVPDTFEDMPEVLAELYEKMV | |
| | | AKGELSRISEPEVPKIPIDYSWAQELGLIRKPAAFISTI | |
| | | SDDRGQELLYAGMPISEVFKEDIGIGGVMSLLWFRRR | |
| | | LPDYASKFLEMVLMLTADHGPAVSGAMNTIITTRAGK | |
| | | DLISSLVAGLLTIGTRFGGALDGAATEFTTAYDKGLSP | |
| | | RQFVDTMRKQNKLIPGIGHRVKSRNNPDFRVELVKDF | |
| | | VKKNFPSTQLLDYALAVEEVTTSKKDNLILNVDGAIAV | |
| | | SFVDLMRSCGAFTVEETEDYLKNGVLNGLFVLGRSIGL | |
| | | IAHHLDQKRLKTGLYRHPWDDITYLVGQEAIQKKRVEI | |
| | | SAGDVSKAKTRS | |
| TGL4 | Yarrowia | MFTSRVSEASTINFIRPTARSHIHFFFAFIAATVHQLLL | 14 |
| | lipolytica | MLYQLLGDGYLKSFVDTGITLAQQSGLSGIVNALTSEA | |
| | | KLRIDKRSIIKKLLEDQENAESYFDWLKASSELDYLLG | |
| | | NQEWKERDECPAYDYEYVRLRLDELRHARTNNDTTRL | |
| | | LYLVRTTWSRNLGNLGDVKLYHNSFTGTKRLIEDYILE | |
| | | CELALNALLAAGNDKIPDQELLTELLNTRKAFGRTALLL | |
| | | SGGGCLGLLHTGVLQALSDTSLLPHVISGSSAGSIMA | |
| | | AGLCIHKDEEHEAFITELMERDFDIFEESGNEDTVLER | |
| | | VSRMLKHGSLLONRYMQDTMRELFGDMTFLEAYNRT | |
| | | RRILNVTVSSAGIYEMPRLLNYLTAPNVLIWSAVCASC | |
| | | SVPLIFNAYTLLEKEPKTGAIQTWNASSLRFIDGSVYA | |
| | | DVPIARLSEMFNVNHFIVSQVNPHVAPFLKLTEDKANP | |
| | | DSVDEIYTLKLWHNFKTLVTDEVMHQLQVLYEFGIFK | |
| | | NLCSKMGGVLSQRYKGDITILPQVHLSELPGILTNPTA | |
| | | AYMKDTNRRGAQATYRKISLIRNHCAIELALDRAIHEL | |
| | | KARMLPSKLGSGRTSPQGTFKHSQSSNQISALKPPSR | |
| | | HMSASSATTAHTRLRNRKSFSHARIKSDAAAVFDKEP | |
| | | IHETPKSSPQSSYVNLHRSASERSRRPKSAFNLGSLPT | |
| | | SPLYHPHLTHSMSMGGANQAPLYNPGRGSVSQNTSP | |
| | | GTKIPGNADPSYFDGPNNVRFHWDSDDDDVRETEFL | |
| | | NNMSSSSSRRVSPVQSRRASVDGLRNSVVSTATSVT | |
| | | DGSVSSRPSRAWESISQLFEGDENCSDSC | |
| FAA1 | Yarrowia | MVGYTISSKPVSVEVGPAKPGETAPRRNVIAKDAPVVF | 15 |
| | lipolytica | PDNDSSLTTVYKLFKKYAEINSERKAMGWRDTIDIHV | |
| | | ETKQVTKVVDGVEKKVPKEWKYFEMGPYKWLSYKEA | |
| | | LKLVHDYGAGLRHLGIKPKEKMHIYAQTSHRWMLSGL | |
| | | ASLSQGIPIVTAYDTLGEEGLTRSLQETNSVIMFTDKA | |
| | | LLSSLKVSLKKGTDLRIIIYGGDLTPDDKKAGNTEIDAI | |
| | | KEIVPDMKIYTMDEVVALGREHPHPVEEVDYEDLAFIM | |
| | | YTSGSTGVPKGVVLQHKQILASVAGVTKIIDRSIIGNT | |
| | | DRLLNFLPLAHIFEFVFEMVTFWWGASLGYGTVKTISD | |
| | | LSMKNCKGDIRELKPTIMVGVPAVWEPMRKGILGKIK | |
| | | ELSPLMQRVFWASFAAKQRLDENGLPGGSILDSLIFK | |
| | | KVKDATGGCLRYVCNGGAPVSVDTOKFITTLICPMLIG | |
| | | CGLTETTANTTIMSPKSYAFGTIGEPTAAVTLKLIDVPE | |
| | | AGYFAENNQGELCIKGNVVMKEYYKNEEETKKAFSDD | |
| | | GYFLTGDIAEWTANGQLRIIDRRKNLVKTQNGEYIALE | |
| | | KLETQYRSSSYVANLCVYADQNRVKPIALVIPNEGPTK | |
| | | KLAQSLGVDSDDWDAVCSNKKVVKAVLKOMLDTGR | |
| | | SLGLSGIELLQGIVLLPGEWTPQNSYLTAAQKLNRKKI | |
| | | VDDNKKEIDECYEQS | |
| FAO1 | Yarrowia | MSDDKHTFDFIIVGGGTAGPTLARRLADAWISGKKLK | 16 |
| | lipolytica | VLLLESGPSSEGVDDIRCPGNWVNTIHSEYDWSYEVD | |
| | | EPYLSTDGEERRLCGIPRGHCLGGSSCLNTSFVIRGTR | |
| | | GDFDRIEEETGAKGWGWDDLFPYFRKHECYVPQGSA | |
| | | HEPKLIDFDTYDYKKFHGDSGPIKVQPYDYAPISKKFS | |
| | | ESLASFGYPYNPEIFVNGGAPQGWGHVVRSTSNGVR | |
| | | STGYDALVHAPKNLDIVTGHAVTKILFEKIGGKQTAVG | |
| | | VETYNRAAEEAGPTYKARYEVVVCCGSYASPQLLMVS | |
| | | GVGPKKELEEVGVKDIILDSPYVGKNLQDHLICGIFVE | |
| | | IKEPGYTRDHQFFDDEGLDKSTEEWKTKRTGFFSNPP | |
| | | QGIFSYGRIDNLLKDDPVWKEACEKQKALNPRRDPM | |
| | | GNDPSQPHFEIWNAELYIELEMTQAPDEGQSVMTVIG | |
| | | EILPPRSKGYVKLLSPDPMENPEIVHNYLQDPVDARVF | |
| | | AAIMKHAADVATNGAGTKDLVKARWPPESKPFEEMSI | |
| | | EEWETYVRDKSHTCFHPCGTVKLGGANDKEAVVDER | |
| | | LRVKGVDGLRVADVSVLPRVPNGHTQAFAYAVGEKAA | |
| | | DLILADIAGKDLRPRI | |
| Linker | Synthetic | GGGGS | 17 |
| | Construct | | |
| Linker | Synthetic | GGGGGGGG | 18 |
| | Construct | | |
| Linker | Synthetic | GGGSGGGSGGGSGGGS | 19 |
| | Construct | | |
| Linker | Synthetic | AEAAAKEAAAKEAAAKEAAAKALEAEAAAKEAAAKEA | 20 |
| | Construct | AAKEAAAKA | |
| Linker | Synthetic | PAPAP | 21 |
| | Construct | | |
| Linker | Synthetic | AEAAAKEAAAKA | 22 |
| | Construct | | |
| Linker | Synthetic | EAAAK | 23 |
| | Construct | | |
| Linker | Synthetic | EAAAKEAAAK | 24 |
| | Construct | | |
| Linker | Synthetic | EAAAKEAAAKEAAAK | 25 |
| | Construct | | |
| Linker | Synthetic | EAAAKEAAAKEAAAKEAAAK | 26 |
| | Construct | | |
| Linker | Synthetic | EAAAKEAAAKEAAAKEAAAKEAAAK | 27 |
| | Construct | | |

TABLE 2
Plasmids

| Plasmids | Description |
|---|---|
| pSL16 | UAS1B8-TEF(136) -hrGFP-CYC, AmpR CEN1 |
| | Leu2 ColE1 |
| pIW245 | UAS1B8-TEF(136) -hrGFP-CYC, AmpR CEN1 |
| | URA3 ColE1 |
| pCRISPRyl_D17 | pCRISPRyl with D17 targeting sgRNA |
| pCRISPRyl_AXP | pCRISPRyl with AXP targeting sgRNA |
| pHR_D17_hrGFP | 1kb_D17_up-UAS1B8-TEF(136)-hrGFP-CYC- |
| | 1kb_D17_down, CEN1 URA3 AmpR ColE1 |
| pHR_AXP_hrGFP | 1kb_AXP_up-UAS1B8-TEF(136)-hrGFP-CYC- |
| | 1kb_AXP_down, CEN1 URA3 AmpR ColE1 |
| pHR_D17_FS001 | 1kb_D17_up-UAS1B8-TEF(136)-TaFAR-BNICL- |
| | CYC-1kb_D17_down, CEN1 URA3 AmpR ColE1 |
| pHR_D17_FS002 | 1kb_D17_up-UAS1B8-TEF(136)-TaFAR-AKL- |
| | CYC-1kb_D17_down, CEN1 URA3 AmpR ColE1 |
| pHR_D17_FS003 | 1kb_D17_up-UAS1B8-TEF(136)-TaFAR-SKL- |
| | CYC-1kb_D17_down, CEN1 URA3 AmpR ColE1 |
| pHR_D17_FS004 | 1kb_D17_up-UAS1B8-TEF(136)-MaACR- |
| | BNICL-CYC-1kb_D17_down, CEN1 URA3 |
| | AmpR ColE1 |
| pHR_D17_FS005 | 1kb_D17_up-UAS1B8-TEF(136)-MmFAR- |
| | BNICL-CYC-1kb_D17_down, CEN1 URA3 |
| | AmpR ColE1 |
| pHR_D17_FS006 | 1kb_D17_up-UAS1B8-TEF(136)-3KAT-GS- |
| | TaFAR-CYC-1kb_D17_down, CEN1 URA3 |
| | AmpR ColE1 |
| pHR_D17_FS007 | 1kb_D17_up-UAS1B8-TEF(136)-3KAT-EF- |
| | TaFAR-CYC-1kb_D17_down, CEN1 URA3 |
| | AmpR ColE1 |
| pHR_D17_FS008 | 1kb_D17_up-UAS1B8-TEF(136)-PTS2-GS- |
| | TaFAR-CYC-1kb_D17_down, CEN1 URA3 |
| | AmpR ColE1 |
| pHR_AXP_FS009 | 1kb_AXP_up-UAS1B8-TEF(136)-3KAT-GS- |
| | TaFAR-CYC-1kb_AXP_down, CEN1 URA3 |
| | AmpR ColE1 |
| pSL16_FS010 | UAS1B8-TEF(136) -GFP-CYC, AmpR CEN1 Leu2 |
| | ColE1 |
| pSL16_FS011 | UAS1B8-TEF(136) -3KAT-GS-GFP-CYC, AmpR |
| | CEN1 Leu2 ColE1 |
| pSL16_FS012 | UAS1B8-TEF(136) -3KAT-GS-TaFAR-CYC, |
| | AmpR CEN1 Leu2 ColE1 |
| pIW245-FS013 | UAS1B8-TEF(136) -IDP3-CYC, AmpR CEN1 |
| | URA3 ColE1 |
| pIW245-FS014 | UAS1B8-TEF(136) -tyPos5-AKL-CYC, AmpR |
| | CEN1 URA3 ColE1 |
| pIW245-FS015 | UAS1B8-TEF(136) -tyPos5-SKL-CYC, AmpR |
| | CEN1 URA3 ColE1 |
| pIW245-FS016 | UAS1B8-TEF(136) -tyPos5-BNICL-CYC, AmpR |
| | CEN1 URA3 ColE1 |
| pIW245-FS017 | UAS1B8-TEF(136) -ANT1-CYC, AmpR CEN1 |
| | Leu2 ColE1 |
| pIW245-FS018 | UAS1B8-TEF(136) -PEX11-CYC, AmpR CEN1 |
| | URA3 ColE1 |
| pIW245-FS019 | UAS1B8-TEF(136) -PEX16-CYC, AmpR CEN1 |
| | URA3 ColE1 |
| pIW245-FS020 | UAS1B8-TEF(136) -ACL-CYC, AmpR CEN1 |
| | URA3 ColE1 |
| pIW245-FS021 | UAS1B8-TEF(136) -TGL4-CYC, AmpR CEN1 |
| | URA3 ColE1 |

TABLE 3
Primers

| | | | SEQ |
|---|---|---|---|
| Primer | Sequence | Gene Targets | ID NO |
| PF101 | CGGGTGGTGGCGCGCCATGGTGTCTATCCCCGAGTACT | TaFAR-F | 28 |
| PF102 | CGGGTGGTGGCGCGCCATGGCCATCCAGCAGGTGCAC | MaACR-F | 29 |
| | CACGCTGACA | | |
| PF103 | CGGGTGGTGGCGCGCCATGGTGTCCATTCCCGAGTACT | MmFAR-F | 30 |
| | ACGA | | |
| PF104 | CGGGTGGTGGCGCGCCATGGACCGACTTAACAACCTC | 3KAT-F | 31 |
| PF105 | GGCAGCGGCGGAGGAGGAAGCGGCGGCGGAGGTTCT | GS-L-Ta-F | 32 |
| | ATGGTGTCTATCCCCGAG | | |
| PF106 | CTTCCTCCTCCGCCGCTGCCACCTCCACCCTCGGCAACA | 3K-GS-L-R | 33 |
| | ACCAGAG | | |
| PF107 | ACATAACTAATTACATGAGGCTAGCTTAGTATCGCATGG | TaFAR-R | 34 |
| | TGGAAGAGGCTCGGAAGTAGGACA | | |
| PF108 | CGGCAGCCGATCCTGCCGATCCCTCGGCAACAACCAGA | 3K-EF-R | 35 |
| | GAAGCGGCA | | |
| PF109 | GGCAGGATCGGCTGCCGGTTCCGGAGAATTCATGGTGT | EF-Ta-F | 36 |
| | CTATCCCCGA | | |
| PF110 | CGGGTGGTGGCGCGCCATGGACCGACTTAACAACCTCG | PTS2-GS-F | 37 |
| | CCACCCAGCTCGGTGGAGGTGGCAGCGGCGGAGGAGG | | |
| | AAGCGGCGGCGGAGGTTCT | | |
| PF111 | AGAACCTCCGCCGCCGCTTCCTCCTCCGCCGCTGCCAC | PTS2-GS-R | 38 |
| | CTCCACCGAGCTGGGTGGCGAGGTTGTTAAGTCGGTCC | | |
| | ATGGCGCGCCACCACCCG | | |
| PF112 | GGTGGAGGTGGCAGCGGCGGAGGAGGAAGCGGCGGC | GS-F-TaFAR | 39 |
| | GGAGGTTCTCGACTTAACAACCTCGCCACCCAGCTCTAA | | |
| | GCTAGCCGGGTGGT | | |
| PF113 | ACCACCCGGCTAGCTTAGAGCTGGGTGGCGAGGTTGTT | TaFAR-R | 40 |
| | AAGTCGAGAACCTCCGCCGCCGCTTCCTCCTCCGCCGC | | |
| | TGCCACCTCCACC | | |
| PF114 | AGCGGCGGAGGAGGAAGCGGCGGCGGAGGTTCTATGG | GS-L-GFP-F | 41 |
| | TGAGTTTCAGAGGCAGC | | |
| PF115 | CATAACTAATTACATGAGGCTAGCTTACACCCACTOGTG | hrGFP-R | 42 |
| | CAGGCTGCC | | |
| PF116 | AGTATAAGAATCATTCAAAATGGTGAGCAAGCAGATCCT | hrGFP-P | 43 |
| | GAAGAACAC | | |
| PF117 | CGGGTGGTGGCGCGCCATGTCCACCACCGCTACTCGAG | IDP3-F | 44 |
| | GC | | |
| PF118 | CGACCACCGCTAGCTTAAGCCAGGTCCTTCTTCAGTCT | IDP3-R | 45 |
| PF119 | CGGGTGGTGGCGCGCCATGGAACTGCTTCCCGACCGC | tyPos5-F | 46 |
| | CAGTC | | |
| PF120 | CGACCACCGCTAGCTTAGAGCTTAGCAGCAACATCGCC | tyPosS-AKL | 47 |
| | TGA | | |
| PF121 | CGACCACCGCTAGCTTACAGCTTGGAAGCAACATCGCC | tyPos5-SKL | 48 |
| | TGA | | |
| PF122 | GAGGCGTCAGGCGATGTTGCTTCTAAGGCCCTGGGCAA | tyPosS- | 49 |
| | GGGAGTGA | BNICL | |
| PF123 | GAGTATAAGAATCATTCAAAATGGTGAGTTTCAGAGGCA | ANT1-F | 50 |
| | GCA | | |
| PF124 | CATAACTAATTACATGAGGCTAGCTTATCCCTTGATCAA | ANTI-R | 51 |
| | GGTGGG | | |
| PF125 | CGGGTGGTGGCGCGCCATGGTGTCTAAGCTGGTTGGA | PEX11-F | 52 |
| | CGGACGA | | |
| PF126 | CGACCACCGCTAGCTTACTAGCAGGTCTTGCATCGCTCC | PEX11-R | 53 |
| | TT | | |
| PF127 | CGGGTGGTGGCGCGCCATGACGGACAAGCTGGTCAAG | PEX16-F | 54 |
| | GTGATGCAGA | | |
| PF128 | ACATAACTAATTACATGAGGCTAGCTTAGAGAGTAGAG | PEX16-R | 55 |
| | GCGGTGAA | | |
| PF129 | CGGGTGGTGGCGCGCCATGATTTCTGCTATTCGTCC | ACL-F | 56 |
| PF130 | CGACCACCGCTAGCTTAGAGCTTGAGGCCAACGA | ACL-R | 57 |
| PF131 | CGGGTGGTGGCGCGCCATGTTCACCTCCAGAGTTTCCG | TGL4-F | 58 |
| | AAGCAA | | |
| PF132 | CGACCACCGCTAGCTTAGCACGAGTCAGAACAGTTCTC | TGL4-R | 59 |
| | GTCC | | |
| PF133 | TTCCGGGTCGGCGCAGGTTGACGTACAGCACATACGAG | IDP3up-F | 60 |
| | GATCGGT | Knockout | |
| PF134 | AACTTGCTATTTCTAGCTCTAAAACACCGATCCTCGTAT | IDP3up-R | 61 |
| | GTGCTG | knockout | |
| PF134 | GGCAGCGGCGGAGGAGGAAGCGGCGGCGGAGGTTCT | GS-MaACR-F | 62 |
| | ATGGCCATCCAGCAGGTGCACCACGC | | |
| PF135 | CGACCACCGCTAGCTTAGTAAGCGGCCTTCTTTCGCTGT | MaACR-R | 63 |
| | CGG | | |
| PF136 | GGCAGCGGCGGAGGAGGAAGCGGCGGCGGAGGTTCT | GS-MmFAR-F | 64 |
| | ATGGTGTCCATTCCCGAGTACTACGA | | |
| PF137 | CGACCACCGCTAGCTTAGTATCGCATGGTGGAAGAGGC | MmFAR-R | 65 |
| | TCGG | | |

TABLE 4
Strains

| Strain | |
|---|---|
| name | Genotype |
| Po1f | (MatA, leu2-270, ura3-302, xpr2-322, axp-2) |
| FS100 | ΔFAA, ΔFAO |
| FS101 | D17:3KAT-GS-TaFAR; ΔFAA, ΔFAO |
| FS102 | D17:3KAT-EF-TaFAR; ΔFAA, ΔFAO |
| FS103 | D17:PTS2-GS-TaFAR; ΔFAA, ΔFAO |
| FS104 | D17:3KAT-GS-MaACR; ΔFAA, ΔFAO |
| FS105 | D17:3KAT-GS-MmFAR; ΔFAA, ΔFAO |
| FS106 | GFP; ΔFAA, ΔFAO |
| FS107 | D17:3KAT-GS-GFP; ΔFAA, ΔFAO |
| FS108 | D17:3KAT-GS-TaFAR; ΔFAA, ΔFAO; pIW245:IDP3; EV(Leu) |
| FS109 | D17:3KAT-GS-TaFAR; ΔFAA, ΔFAO; pIW245:Pos5 − AKL; |
| | EV(Leu) |
| FS110 | D17:3KAT-GS-TaFAR; ΔFAA, ΔFAO; pIW245:Pos5 − SKL; |
| | EV(Leu) |
| FS111 | D17:3KAT-GS-TaFAR; ΔFAA, ΔFAO; pIW245:Pos5 − |
| | BNICL; EV(Leu) |
| FS112 | D17:3KAT-GS-TaFAR; ΔFAA, ΔFAO; pIW245:ANT1; |
| | EV(Leu) |
| FS113 | D17:3KAT-GS-TaFAR; ΔFAA, ΔFAO; pIW245:ANT1; |
| | pSL16:Pos5 + SKL |
| FS114 | D17:3KAT-GS-TaFAR; ΔFAA, ΔFAO; pIW245-PEX11; |
| | EV(Leu) |
| FS115 | D17:3KAT-GS-TaFAR; ΔFAA, ΔFAO; pIW245:PEX16; |
| | EV(Leu) |
| FS116 | D17:3KAT-GS-TaFAR; ΔFAA, ΔFAO; pIW245:ACL; EV(Leu) |
| FS117 | D17:3KAT-GS-TaFAR; ΔFAA, ΔFAO; pIW245:TGL4; |
| | EV(Leu) |
| FS118 | D17:3KAT-GS-TaFAR; AXP:3KAT-GS-TaFAR; ΔFAA, |
| | ΔFAO; pIW245:Pos5 − SKL; EV(Leu) |
| FS119 | D17:3KAT-GS-TaFAR; AXP:3KAT-GS-TaFAR; |
| | pSL16:3KAT-GS-TaFAR; ΔFAA, ΔFAO; |
| | pIW245:Pos5 − SKL |
| FS120 | D17:3KAT-GS-TaFAR; ΔIDP3, ΔFAA, ΔFAO |

TABLE 5
Codon optimized genes

| Synthesized | | SEQ ID |
|---|---|---|
| genes | Sequence (5′-3′) | NO: |
| TaFAR1 | ATGGTGTCTATCCCCGAGTACTACGAGGGAAAGAACATTCTGCT | 66 |
| | GACCGGAGCTACCGGTTTCATGGGCAAGGTGCTGCTGGAGAAG | |
| | CTGCTGCGATCCTGTCCCAAGGTCAAGGCCGTCTACGTGCTGGT | |
| | GCGACACAAGGCTGGACAGACCCCTGAGGCTCGAATTGAGGAG | |
| | ATTACCAACTGCAAGCTGTTCGACCGACTGCGAGACGAGCAGCC | |
| | CGACTTCAAGGCTAAGATCATTGTGATCACCTCTGAGCTGACCCA | |
| | GCCTGAGCTGGACCTGTCTGAGCCCATTAAGGAGAAGCTGATCG | |
| | AGCGAATTAACATCATTTTOCACTGTGCCGCTACCGTCCGATTCA | |
| | ACGAGACCCTGCGAGACGCTGTGCAGCTGAACGTCACCGCTACC | |
| | CAGCAGCTGCTGTTCCTGGCTCAGCGAATGAAGAACCTGGAGGT | |
| | GTTCATGCACGTCTCTACCGCTTACGCCTACTGCAACCGAAAGCA | |
| | GATCGAGGAGATTGTGTACCCTCCTCCCGTCGACCCCAAGAAGC | |
| | TGATCGACTCCCTGGAGTGGATGGACGACGGACTGGTGAACGA | |
| | CATCACCCCCAAGCTGATTGGTGACCGACCCAACACCTACACCTA | |
| | CACCAAGGCTCTGGCCGAGTACGTGGTCCAGCAGGAAGGCGCT | |
| | AAGCTGAACACCGCCATCATTCGACCCTCTATCGTGGGTGCTTCC | |
| | TGGAAGGAGCCCTTCCCCGGCTGGATTGACAACTTCAACGGACC | |
| | CTCTGGTCTGTTCATCGCCGCTGGAAAGGGCATTCTGCGAACCA | |
| | TGCGAGCCTCTAACTCTGCTGTGGCTGACCTGGTGCCTGTGGAC | |
| | GTGGTCGTGAACACCACCCTGGCTGCTGCTTGGTACTCTGGTGT | |
| | GAACCGACCCCGAAACGTCATGATTTACAACTGTACCACCGGCG | |
| | GAACCAACCCTTTCCACTGGGGCGAGGTCGGATACCACATCAAC | |
| | CTGAACTTCAAGATTAACCCCCTGGAGAACGCCGTCCGACACCC | |
| | CAACTGCTCTCTGCAGTCCAACCCCCTGCTGCACCAGTACTGGA | |
| | CCGCTGTGTCTCACACCATGCCTGCCTTCCTGCTGGACCTGCTGC | |
| | TGCGACTGACCGGACACAAGCCCTGGATGATGAAGACCATCACC | |
| | CGACTGCACAAGGCTATGATGCTGCTGGAGTACTTCACCTCTAAC | |
| | TCCTGGATTTGGAACACCGAGAACATGACCATGCTGATGAACCA | |
| | GCTGAACCCCGAGGACAAGAAGACCTTCAACTTCGACGTGCGAC | |
| | AGCTGCACTGGGCCGAGTACATGGAGAACTACTGTATGGGCACC | |
| | AAGAAGTACGTCCTGAACGAGGAGATGTCTGGACTGCCCGCTGC | |
| | CCGAAAGCACCTGAACAAGCTGCGAAACATCCGATACGGCTTCA | |
| | ACACCGTGCTGGTCATCCTGATTTGGCGAATCTTCATTGCTCGAT | |
| | CCCAGATGGCCCGAAACATTTGGTACTTCGTCGTGTCTCTGTGCT | |
| | ACAAGTTCCTGTCCTACTTCCGAGCCTCTTCCACCATGCGATACT | |
| | AA | |
| MaACR | ATGGCCATCCAGCAGGTGCACCACGCTGACACCTCTTCCTCTAA | 67 |
| | GGTCCTGGGTCAGCTGCGAGGCAAGCGAGTGCTGATTACCGGC | |
| | ACCACCGGATTCCTGGGAAAGGTGGTCCTGGAGCGACTGATCCG | |
| | AGCCGTCCCCGACATCGGCGCTATTTACCTGCTGATTCGAGGAA | |
| | ACAAGCGACACCCCGACGCCCGATCCCGATTCCTGGAGGAGATC | |
| | GCTACCTCCTGTGTGTTCGACCGACTGCGAGAGGCCGACTCTGA | |
| | GGGTTTCGACGCTTTCCTGGAGGAGCGAATCCACTGCGTGACCG | |
| | GAGAGGTGACCGAGGCTGGTTTCGGAATTGGTCAGGAAGACTAC | |
| | CGAAAGCTGGCCACCGAGCTGGACGCTGTCATTAACTOCGCCGC | |
| | TTCTGTGAACTTCCGAGAGGAGCTGGACAAGGCCCTGGCTATCA | |
| | ACACCCTGTGTCTGCGAAACATTGCCGGAATGGTCGACCTGAAC | |
| | CCCAAGCTGGCTGTGCTGCAGGTCTCCACCTGCTACGTGAACGG | |
| | AATGAACTCCGGTCAGGTCACCGAGTCTGTGATTAAGCCTGCTG | |
| | GAGAGGCTGTCCCTCGATCTCCCGACGGTTTCTACGAGATCGAG | |
| | GAGCTGGTCCGACTGCTGCAGGACAAGATTGAGGACGTGCAGG | |
| | CCCGATACTCCGGCAAGGTCCTGGAGCGAAAGCTGGTGGACCT | |
| | GGGTATTCGAGAGGCTAACCGATACGGCTGGTCTGACACCTACA | |
| | CCTTCACCAAGTGGCTGGGCGAGCAGCTGCTGATGAAGGCCCTG | |
| | AACGGACGAACCCTGACCATCCTGCGACCCTCCATCATTGAGTC | |
| | CGCTCTGGAGGAGCCTGCTCCTGGTTGGATTGAGGGAGTGAAG | |
| | GTCGCCGACGCTATCATTCTGGCCTACGCTCGAGAGAAGGTGAC | |
| | CCTGTTCCCCGGAAAGCGATCCGGCATCATTGACGTGATCCCCG | |
| | TCGACCTGGTGGCCAACTCCATCATTCTGTCTCTGGCTGAGGCTC | |
| | TGGGAGAGCCTGGACGACGACGAATTTACCAGTGTTGCTCCGGC | |
| | GGAGGTAACCCCATCTCTCTGGGAGAGTTCATTGACCACCTGAT | |
| | GGCCGAGTCCAAGGCTAACTACGCCGCTTACGACCACCTGTTCT | |
| | ACCGACAGCCCTCTAAGCCCTTCCTGGCCGTCAACCGAGCTCTG | |
| | TTCGACCTGGTCATCTCCGGAGTGCGACTGCCCCTGTCTCTGAC | |
| | CGACCGAGTGCTGAAGCTGCTGGGTAACTCCCGAGACCTGAAGA | |
| | TGCTGCGAAACCTGGACACCACCCAGTCTCTGGCCACCATCTTC | |
| | GGATTCTACACCGCTCCCGACTACATTTTCCGAAACGACGAGCTG | |
| | ATGGCCCTGGCTAACCGAATGGGAGAGGTCGACAAGGGTCTGTT | |
| | CCCCGTGGACGCCCGACTGATCGACTGGGAGCTGTACCTGCGAA | |
| | AGATTCACCTGGCCGGCCTGAACCGATACGCTCTGAAGGAGCGA | |
| | AAGGTGTACTCTCTGAAGACCGCCCGACAGCGAAAGAAGGCCGC | |
| | TTACTAA | |
| MmFAR | ATGGTGTCCATTCCCGAGTACTACGAGGGCAAGAACATCCTGCT | 68 |
| | GACCGGTGCTACCGGCTTCCTGGGAAAGGTGCTGCTGGAGAAG | |
| | CTGCTGCGATCTTGTCCCCGAGTCAACTCCGTCTACGTGCTGGTC | |
| | CGACAGAAGGCCGGACAGACCCCTCAGGAGCGAGTCGAGGAGA | |
| | TTCTGTCTTCCAAGCTGTTCGACCGACTGCGAGACGAGAACCCC | |
| | GACTTCCGAGAGAAGATCATTGCTATCAACTCTGAGCTGACCCA | |
| | GCCCAAGCTGGCCCTGTCCGAGGAAGACAAGGAGATCATTATCG | |
| | ACTCTACCAACGTGATTTTCCACTGTGCCGCTACCGTCCGATTCA | |
| | ACGAGAACCTGCGAGACGCTGTGCAGCTGAACGTCATTGCCACC | |
| | CGACAGCTGATCCTGCTGGCCCAGCAGATGAAGAACCTGGAGGT | |
| | GTTCATGCACGTCTCCACCGCTTACGCCTACTGCAACCGAAAGCA | |
| | CATTGACGAGGTGGTCTACCCTCCTCCCGTGGACCCCAAGAAGC | |
| | TGATCGACTCTCTGGAGTGGATGGACGACGGTCTGGTCAACGAC | |
| | ATTACCCCCAAGCTGATCGGCGACCGACCCAACACCTACATTTAC | |
| | ACCAAGGCTCTGGCCGAGTACGTGGTCCAGCAGGAAGGCGCTA | |
| | AGCTGAACGTGGCCATTGTCCGACCCTCTATCGTGGGAGCTTCC | |
| | TGGAAGGAGCCTTTCCCTGGATGGATCGACAACTTCAACGGTCC | |
| | CTCCGGCCTGTTCATTGCCGCTGGCAAGGGAATCCTGCGAACCA | |
| | TGCGAGCCTCTAACAACGCTCTGGCTGACCTGGTGCCTGTGGAC | |
| | GTGGTCGTGAACACCTCCCTGGCTGCTGCTTGGTACTCTGGTGT | |
| | GAACCGACCCCGAAACATTATGGTCTACAACTGCACCACCGGAT | |
| | CCACCAACCCCTTCCACTGGGGTGAGGTGGAGTACCACGTCATC | |
| | TCTACCTTCAAGCGAAACCCCCTGGAGCAGGCTTTCCGACGACC | |
| | CAACGTGAACCTGACCTCCAACCACCTGCTGTACCACTACTGGAT | |
| | TGCCGTCTCTCACAAGGCTCCCGCCTTCCTGTACGACATCTACCT | |
| | GCGAATGACCGGACGATCTCCCCGAATGATGAAGACCATTACCC | |
| | GACTGCACAAGGCTATGGTGTTCCTGGAGTACTTCACCTCTAACT | |
| | CCTGGGTGTGGAACACCGACAACGTCAACATGCTGATGAACCAG | |
| | CTGAACCCCGAGGACAAGAAGACCTTCAACATTGACGTGCGACA | |
| | GCTGCACTGGGCCGAGTACATCGAGAACTACTGTATGGGAACCA | |
| | AGAAGTACGTCCTGAACGAGGAGATGTCTGGTCTGCCTGCTGCT | |
| | CGAAAGCACCTGAACAAGCTGCGAAACATTCGATACGGCTTCAA | |
| | CACCATCCTGGTGATTCTGATCTGGCGAATTTTCATCGCTCGATC | |
| | TCAGATGGCCCGAAACATCTGGTACTTCGTCGTGTCTCTGTGCTA | |
| | CAAGTTCCTGTCCTACTTCCGAGCCTCTTCCACCATGCGATACTA | |
| | A | |
| Truncated | ATGGAACTGCTTCCCGACCGCCAGTCGCCCCACTACGTCCAGGA | 69 |
| Pos5 or | GTCGACCTCTCTGTCATCTCTGGTGTGGGACAAGCCTCTGGAAA | |
| tyPos5 | ACGTTCTGATCGTCAAAAAACCCTGGGACCACAATGTGCGCGAG | |
| | TCGCTCATCCAGATGGCATCTCACATCCAGCGCCGGTACCCCCG | |
| | AGTCAACATTCTGGTGGAGGAACATGTGGCCGACGAGGTCCAGA | |
| | AGCAGATTGGAGCCGCAGGCGTGACCGCCATCCACACGGGGCC | |
| | AGGAGAGGTGCTGAGAAACAAGACGGATCTGCTCGTGACTCTGG | |
| | GAGGCGACGGAACTATTCTACATGCCACCTCCATGTTTGCTTCCG | |
| | GAGAAGTGCCGCCGGTGCTGTCCTTTTCGCTGGGGACTCTGGGT | |
| | TTCCTGCTGCCGTTTGATTTCAAGGACTTCAAAACTGCATTCGAC | |
| | ATGGTGTACTCGTCGCAGGCCTCGGTGGTCAACCGCGCCCGCCT | |
| | AGCATGTCAGAAAATGTCCATTCGCAAGGAAATCACCCACTTGCC | |
| | CTCCCAATCGCACATTGAACACAACTCAACCCATGTCTACGGCAA | |
| | TCCCGACGACTACAATCTTAGCCCACTAACCTACGCCATGAACGA | |
| | CATCAACATCCACCGTGGAGCTGAGCCGCATCTCACCAAGCTCG | |
| | ACATCCACGTTGACGGCGAGTTCATCACCCGAGCCATTGCTGAC | |
| | GGTGTCACCATCGCCACACCCACGGGCTCCACGGCCTACTCGCT | |
| | GTCGTCTGGCGGCTCCATTGTGCATCCCCGAGTCGCCTGCATTC | |
| | TGCTGACCCCCATCTGTCCGCGATCGCTGTCATTCCGGCCTCTCA | |
| | TTTTCCCAGCCACCTCCAAAATATGCATCACCGCCTCGTCCGAAT | |
| | CTCGAGGTAGAGGCGCCGAGCTGTCTGTCGACGGAATCGCCAA | |
| | GGGTCTGGTTCGACCCAGCGACAAGATTCTGGTCGAAAGCGAAA | |
| | CCGGCCACAACTCGGGCATCTGGTGCGTGGCCAAGACAGACAG | |
| | AGACTGGGTCAGTGGCCTCAACGGGTTACTGGGCTTCAATAGCA | |
| | GTTTTGGCAAGGGGGGGGAGGCGTCAGGCGATGTTGCTTAA | |

TABLE 6
Fatty alcohol distribution data from bioreactor run

| | Fatty Alcohol | Fatty Alcohol |
|---|---|---|
| Alcohols | Titer (g/L) | Distribution (%) |
| Hexadecanol (C16:0) | 2.53 ± 0.05 | 91 |
| Octadecanol (C18:0) | 0.23 ± 0.006 | 9 |
| Total | 2.77 ± 0.05 | 100% |

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A recombinant Yarrowia lipolytica, comprising a heterologous polynucleotide encoding a fusion protein, wherein the fusion protein comprises a first amino acid sequence at least 80% identical to the amino acid sequence of 3-ketoacyl CoA thiolase (3KAT) (SEQ ID NO: 1) and a second amino acid sequence at least 80% identical to the amino acid sequence of a fatty acyl-CoA reductase (FAR).

2. The recombinant Yarrowia lipolytica of claim 1, wherein the first amino acid sequence is the amino acid sequence of the 3KAT (SEQ ID NO: 1) and the second amino acid sequence is the amino acid sequence of the FAR.

3. The recombinant Yarrowia lipolytica of claim 1, wherein the FAR is selected from the group consisting of TaFAR (SEQ ID NO: 2), MaACR (SEQ ID NO: 3) and MmFAR (SEQ ID NO: 4).

4. The recombinant Yarrowia lipolytica of claim 1, wherein the fusion protein further comprises a linker between the first amino acid sequence and the second amino acid sequence.

5. The recombinant Yarrowia lipolytica of claim 1, wherein the recombinant Yarrowia lipolytica has knockout of fatty acyl-CoA synthetase (FAA1) (SEQ ID NO: 15), fatty alcohol oxidase (FAO1) (SEQ ID NO: 16), or a combination thereof.

6. The recombinant Yarrowia lipolytica of claim 1, wherein the recombinant Yarrowia lipolytica expresses the fusion protein in one or more peroxisomes in the recombinant Yarrowia lipolytica and generates one or more fatty alcohols in the one or more peroxisomes.

7. The recombinant Yarrowia lipolytica of claim 6, wherein the one or more fatty alcohols comprise a C16:0 fatty alcohol, a C18:0 fatty alcohol, or a combination thereof.

8. The recombinant Yarrowia lipolytica of claim 6, wherein the recombinant Yarrowia lipolytica further expresses a recombinant NADH kinase having an amino acid sequence at least 80% identical to SEQ ID NO: 8 in the one or more peroxisomes.

9. The recombinant Yarrowia lipolytica of claim 6, wherein the recombinant Yarrowia lipolytica overexpresses an endogenous protein selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), and triacylglycerol lipase (TGL4) (SEQ ID NO: 14).

10. A method for producing one or more fatty alcohols, comprising:
(a) growing a recombinant Yarrowia lipolytica in a culture medium, wherein the recombinant Yarrowia lipolytica comprises a heterologous polynucleotide encoding a fusion protein, wherein the fusion protein comprises a first amino acid sequence at least 80% identical to the amino acid sequence of 3-ketoacyl CoA thiolase (3KAT) (SEQ ID NO: 1) and a second amino acid sequence at least 80% identical to the amino acid sequence of a fatty acyl-CoA reductase (FAR);
(b) expressing the fusion protein in one or more peroxisomes in the recombinant Yarrowia lipolytica; and
(c) generating one or more fatty alcohols by the recombinant Yarrowia lipolytica, whereby the one or more fatty alcohols are produced in the culture medium.

11. The method of claim 10, wherein the first amino acid sequence is the amino acid sequence of the 3KAT (SEQ ID NO: 1) and the second amino acid sequence is the amino acid sequence of the FAR.

12. The method of claim 10, wherein the FAR is selected from the group consisting of TaFAR (SEQ ID NO: 2), MaACR (SEQ ID NO: 3) and MmFAR (SEQ ID NO: 4).

13. The method of claim 10, wherein the fusion protein further comprises a linker between the first amino acid sequence and the second amino acid sequence.

14. The method of claim 10, wherein the recombinant Yarrowia lipolytica has knockout of fatty acyl-CoA synthetase (FAA1) (SEQ ID NO: 15), fatty alcohol oxidase (FAO1) (SEQ ID NO: 16), or a combination thereof.

15. The method of claim 10, further comprising expressing a recombinant NADH kinase in the one or more peroxisomes by the recombinant Yarrowia lipolytica, wherein the recombinant NADH kinase comprises an amino acid sequence at least 80% identical to SEQ ID NO: 8.

16. The method of claim 10, further comprising overexpressing an endogenous protein selected from the group consisting of isocitrate dehydrogenase enzyme (IDP3) (SEQ ID NO: 9), ADP/ATP translocase 1 (ANT1) (SEQ ID NO: 10), peroxisome biogenesis factor 11 (PEX11) (SEQ ID NO: 11), peroxisome biogenesis factor 16 (PEX16) (SEQ ID NO: 12), ATP citrate lyase (ACL) (SEQ ID NO: 13), and triacylglycerol lipase (TGL4) (SEQ ID NO: 14).

17. The method of claim 10, further comprising adding dodecane to the culture medium before the growing, whereby the culture medium becomes biphasic.

18. The method of claim 10, wherein the recombinant Yarrowia lipolytica is grown in a continuous culture.

19. (canceled)

20-22. (canceled)

23. A method for preparing a recombinant Yarrowia lipolytica, comprising introducing a heterologous polynucleotide into Yarrowia lipolytica, wherein the heterologous polynucleotide encodes a fusion protein, and wherein the fusion protein comprises a first amino acid sequence at least 80% identical to the amino acid sequence of 3-ketoacyl CoA thiolase (3KAT) (SEQ ID NO:1) and a second amino acid sequence at least 80% identical to the amino acid sequence of a fatty acyl-CoA reductase (FAR), whereby a recombinant Yarrowia lipolytica is prepared.

24-31. (canceled)

32. A recombinant Yarrowia lipolytica prepared by the method of claim 23.
